(19) United States
(12) Patent Application Publication
(10) Pub. No.: US 2024/0425931 A1
(43) Pub. Date: Dec. 26, 2024

(54) DNA METHYLATION BARRIERS

(71) Applicants: Li Liu, Scottsdale, AZ (US); Jingmin Shu, Phoenix, AZ (US)

(72) Inventors: Li Liu, Scottsdale, AZ (US); Jingmin Shu, Phoenix, AZ (US)

(73) Assignee: Arizona Board of Regents on Behalf of Arizona State University, Tempe, AZ (US)

(21) Appl. No.: 18/749,335

(22) Filed: Jun. 20, 2024

Related U.S. Application Data
(60) Provisional application No. 63/522,078, filed on Jun. 20, 2023.

(51) Int. Cl. C12Q 1/6886

(52) U.S. Cl. CPC C12Q 1/6886; C12Q 2600/118; C12Q 2600/154

(57) ABSTRACT

Hypermethylation near promoter regions prevents expression and is associated with pathogenesis in diseases. Hundreds of methylation barriers contain a 41-bp DNA sequence motif that is common across a variety of genes. The present disclosure provides computational and experimental methods to investigate methylation barriers. More than 500 methylation barriers are identified that shared a common 41-bp sequence motif (MB-41).

### CROSS REFERENCE TO RELATED APPLICATIONS

This application claims the priority from U.S. Provisional Application No. 63/522,078, filed Jun. 20, 2023, entitled “DNA METHYLATION BARRIERS”, the contents of which are hereby incorporated by reference in their entirety.

### ACKNOWLEDGEMENT OF GOVERNMENT SUPPORT

This invention was made with government support under R01-LM013438 awarded by the National Institutes of Health. The government has certain rights in the invention.

### SEQUENCE LISTING

The present application contains a Sequence Listing which has been submitted electronically in .XML format and is hereby incorporated herein by reference in its entirety. Said computer readable file, was created on Jun. 19, 2024 is named 055743_802486_SquenceListing.xml and is 10 kilobytes in size.

### BACKGROUND

DNA methylation is a repressive epigenetic modification of vertebrate genomes, and is essential to transcriptional regulation, genome stability, and other cellular functions. In a normally differentiated cell, a majority of the genome is densely methylated, but regions known as CpG islands (CGIs) near gene promoters are unmethylated. Maintaining the boundary between unmethylated promoter-associated CGIs and adjacent methylated regions is believed to be crucial to normal cell function, and loss of segregation can lead to disease. Yet the way in which promoter-associated CGIs in their normal genomic context resist evasion of the DNA methylation machinery and whether these mechanisms are evolutionarily conserved remains enigmatic. A need exists for methods and systems/kits for identifying aberrant methylation patterns of CGIs that can predict the risk or presence of disease.

### SUMMARY OF THE INVENTION

In various aspects the present disclosure encompasses methods, systems, or kits for monitoring and/or treating a cancer in a subject in need thereof, by first detecting the absence of a functional methylation barrier in a subject's genome, and/or by first detecting presence of the cancer or an increased risk of cancer in the subject.

In one aspect, the present disclosure encompasses a method comprises analyzing a test genomic sequence obtained from the subject to determine the presence or absence of a protective methylation barrier for at least one CpG island in the test genomic sequence. The absence of a protective methylation barrier for at least one CpG island in the test genomic sequence is indicative of increased cancer risk in the subject. In certain aspects, the cancer is colorectal cancer or an esophageal cancer. Analyzing the genomic sequence may comprise: (a) identifying in the sequence the occurrence of at least one PIR trio comprising a combination of a promoter, a CpG island and a repeat sequence in close vicinity; (b) for each PIR trio identified in (a), determining the methylation of any one of or any combination of the promoter, the CpG island and the repeat sequence; (c) comparing the methylation from (b) with the methylation of the one of, or the combination of any of the promoter, the CpG island and the repeat sequence in the PIR trio in a control genomic sample to identify differentially methylated PIRs (dmPIR); (d) aggregating dmPIRs around the same TSS to locate the genomic region of interest, i.e., dmPIR-tagged region and (d) detecting the absence of a protective methylation barrier in the test genomic sequence, i.e., if methylation spreading in the dmPIR-tagged region in the test genomic sequence is greater than that in the control genomic sample, wherein the absence of the methylation barrier is indicative of increased cancer risk in the subject. In one aspect, the cancer risk is a risk for colorectal cancer or an esophageal cancer. The method may further comprise obtaining or having obtained a biological sample from the subject, wherein the biological sample contains the test genomic sequence. The cancer may be a colorectal cancer or an esophageal cancer, such as a colonic adenocarcinoma or an esophageal squamous cell carcinoma. In another aspect, the at least one CpG island is promoter-associated. In another aspect, the protective methylation barrier comprises a nucleotide sequence displaying a specific pattern of SEQ ID NO: 1 encoding the 41-bp motif (MB-41). In one aspect, the “B” in SEQ ID NO: 1 is selected from C, G and T. In another aspect, the “S” in SEQ ID NO: 1 is selected from C and G. In another aspect, the “K” in SEQ ID NO: 1 is selected from T and G. In another aspect, the “Y” in SEQ ID NO: 1 is selected from C and T. In another aspect, the at least one CpG island is associated with the nucleotide sequence of the P16 (a.k.a., CDKN2) tumor suppressor having the sequence of SEQ ID NO: 2. In another aspect, the at least one CpG island is associated with the nucleotide sequence of other tumor suppressor genes.

Certain aspects of the present disclosure also provide methods for identifying a compromised methylation barrier in a genome of a subject, for example a method that comprises: (a) analyzing a genomic sample of the subject to identify at least one transcription start site (TSS); (b) for each TSS, searching a region including and/or within the TSS and about ±2000 bps flanking the TSS region for CpG island and nucleic acid repeats, wherein an occurrence of a combination of a promoter sequence, a CGI, and a repeat sequence comprises a promoter-CpG island (island)-repeat (PIR) trio indicative of a candidate methylation barrier; and (c) using whole or partial genome sequencing to determine whether any one or any combination of the promoter, the CpG island and the repeat of the PIR trio have increased methylation compared to that obtained from a normal control sample, wherein increased methylation of any one or any combination of the promoter, the CpG island and the repeat of the PIR trio is indicative of the presence of a compromised methylation barrier in the genome of the subject. In some aspect, the method further comprises obtaining or having obtained a biological sample from the subject and the biological sample provides the genome of the subject. In one aspect, the presence of the compromised methylation barrier is indicative of a colorectal cancer in the subject. In another aspect, the presence of the compromised methylation barrier is indicative of a colonic adenocarcinoma in the subject. In some aspect, at least one CpG island is promoter-associated. In another aspect, the compromised methylation barrier comprises a nucleotide sequence of SEQ ID NO: 1 [41-bp motif (MB-41)]. In one aspect, the “B” in SEQ ID NO: 1 is selected from C, G and T. In another aspect, the “S” in SEQ ID NO: 1 is selected from C and G. In another aspect, the “K” in SEQ ID NO: 1 is selected from T and G. In another aspect, the “Y” in SEQ ID NO: 1 is selected from C and T. In another aspect, the at least one CpG island is associated with the nucleotide sequence of the P16 tumor suppressor having the sequence of SEQ ID NO: 2. In another aspect, the at least one CpG island is associated with the nucleotide sequence of another tumor suppressor gene.

Certain aspects of the present disclosure also provide system or kit, such as a computerized system for identifying the presence of a cancer or an increased risk of a cancer in a subject. In one aspect, the system or kit comprises (a) a biological sample wherein a genomic DNA sequence can be obtained; (b) a reagent selected from one or more from the group consisting of a DNA extraction reagent, a bisulfite treatment reagent, a primer, a PCR reagent, a reagent for next-generation sequence, and a reagent to measure methylation level in the biological sample; (c) a control sample comprising LNCaP cell line DNA and a P16 gene primer; (d) instructions for a user. In one aspect, the instructions comprise the steps of (i) processing the biological sample to a bisulfite conversion process and a sequencing preparation step; (ii) comparing the processed biological sample with the control sample to ensure proper implementation of bisulfite conversion and sequencing preparation; (iii) obtaining the genomic DNA sequence information from the processed biological sample, wherein the genomic DNA sequence information comprises a sequence of at least one transcription start site (TSS); (iv) searching a region within the TSS and about ±200 bps flanking the TSS region for each occurrence of a combination of a promoter sequence, a CpG island, and a repeat sequence, wherein the combination is a promoter-CpG island (island)-repeat (PIR) trio indicative of methylation barrier; (v) determining methylation of each occurrence of the combination of the promoter sequence, the CpG island and the repeat sequence against that of the control sample; and (vi) identifying the presence of the cancer or increased risk of the cancer in the subject when the subject possesses an increased methylation or a compromised methylation barrier comparing with that of the control sample. In one aspect, the system or kit comprises (a) a first database configured to store information regarding a genome of the subject obtained by whole or partial genome sequencing, wherein the information comprises a sequence of at least one transcription start site (TSS); (b) a processor configured to perform (i) instructions for searching a region including the TSS and about ±2000 bps flanking the TSS region for each occurrence of a combination of a promoter sequence, a CpG island, and a repeat sequence, wherein the combination is a promoter-CpG island (island)-repeat (PIR) trio indicative of a candidate methylation barrier; (ii) instructions to identify dmPIRs by determining the relative methylation of any one of, or any combination of the promoter, the CpG island and the repeat of the PIR trio compared to the methylation the one of, or the combination of the promoter, the CpG island and the repeat of the PIR trio obtained from a normal control sample; (iii) instructions for locating dmPIR-tagged regions by aggregating dmPIRs around the same TSS; and (iv) instructions for determining methylation spreading in dmPIR-tagged regions, wherein increased methylation of the one of, or the combination of any of the promoter, the CpG island and the repeat of the PIR trio compared to the normal control sample is indicative of the presence of a compromised methylation barrier in the genome of the subject and indicative of the presence of a cancer or increased risk of cancer in the subject. In another aspect, the presence of the compromised methylation barrier is indicative of a colorectal cancer or an esophageal cancer in the subject, and the presence of the compromised methylation barrier is indicative of a colonic adenocarcinoma or an esophageal squamous cell carcinoma in the subject. In some aspect, the at least one CpG island is promoter-associated. In yet another aspect, the compromised methylation barrier comprises a nucleotide sequence of SEQ ID NO: 1 [41-bp motif (MB-41)]. In one aspect, the “B” in SEQ ID NO: 1 is selected from C, G and T. In another aspect, the “S” in SEQ ID NO: 1 is selected from C and G. In another aspect, the “K” in SEQ ID NO: 1 is selected from T and G. In another aspect, the “Y” in SEQ ID NO: 1 is selected from C and T. In some aspect, the at least one CpG island is associated with the nucleotide sequence of the P16 tumor suppressor having the sequence of SEQ ID NO: 2. In another aspect, the at least one CpG island is associated with the nucleotide sequence of another tumor suppressor gene.

The foregoing is intended to be illustrative and is not meant in a limiting sense. Many features and sub-combinations of the present inventive concept may be made and will be readily evident upon a study of the following specification and accompanying drawings comprising a part thereof. These features and sub-combinations may be employed without reference to other features and sub-combinations.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Aspects of the present disclosure are illustrated by way of example in which like reference numerals indicate similar elements and in which:

FIGS. 1A-1G depict genome-wide screening for methylation barriers and sequence signatures. FIG. 1A is a schematic illustration of two PIR trios found in a ±2 kbps TSS-flanking region. The ±200 bps TSS-flanking region is a proxy for the promoter. There are two repeats and one CpG island in this region. Each trio comprises a promoter, a CpG island, and a repeat. FIG. 1B is a schematic illustration of a dmPIR-tagged region. Each bar represents a CpG site with the height corresponding to methylation level. The promoter is protected from DNA methylation spreading from surrounding repetitive elements in normal samples (upper panel). The protection is compromised in cancer samples (lower panel). FIG. 1C are split violin plots showing distribution of methylation by location (repeats, CpG islands, and promoters) and sample type (normal and cancer) in a WGBS data set. In the 2,252 dmPIRs, repeats were hypermethylated in both normal and cancer samples. CpG islands and TSSs were unmethylated in normal samples but became methylated in cancers. FIG. 1D is transition of methylation level in regions tagged by the 2,252 dmPIRs showing methylation spreading. For each dmPIR-tagged region, the repeat was divided into 3 equal-sized windows; the promoter was divided into 3 equal-sized windows; and the segment in between was divided into 14 equal-sized windows. Mean methylation level in each window across all dmPIR-tagged regions was plotted. Error bars represent standard deviations (SD). FIG. 1E is sequence logo of the MB-41 motif. FIG. 1F are Boxplots showing the q values representing false discovery rate of MB-41 matches was significantly higher in non-dmPIR-tagged unprotected regions than in dmPIR-tagged protected regions. FIG. 1G are block diagram (upper panel) showing the five functional sites (FI-FV) in the 235 bps chicken HS4 core element, and sequence alignment (lower panel) showing the FIII site and the flanking region (red line) matched to the MB-41 motif.

FIGS. 2A-2B depict DNA methylation of protected fragments tagged by dmPIRs, and unprotected fragments tagged by non-dmPIRs. Each region was divided into 20 sliding windows. Mean methylation level within each window was calculated. FIG. 2A depicts data as mean±SD across all dmPIR-tagged protected regions. FIG. 2B depicts data across all non-dmPIR-tagged unprotected regions (B).

FIG. 3A-3G depict validation of dmPIRs. FIG. 3A depicts transition of methylation level in regions tagged by the 620 dmPIRs reproduced in an independent RRBS data set. Normal and cancer samples were from the RRBS data set. FIG. 3B depicts violin plots show methylation of repeats, CpG islands, and promoters in 2,252 dmPIRs in ENCODE normal samples. It confirmed hypermethylation of repeats and hypomethylation of CpG islands and promoters. FIG. 3C depicts transition of methylation level in regions tagged by the 2,252 dmPIRs in ENCODE normal samples. FIG. 3D depicts structure of the TSS-flanking region (Chr9: 21,976,749-21,973,876) of the P16 gene. The TSS is at the 5′ end of the first exon (open rectangle) and is inside the CpG island (green rectangle). The promoter is juxtaposed between three upstream repeats and one downstream repeat (black rectangles). FIG. 3E depicts DNA methylation patterns of the P16 TSS-flanking region displayed at the same scale as FIG. 3D. The region was divided into 40 sliding windows (window size=200 bps, step size=100 bps). Mean methylation level within each window was plotted. All samples in the WGBS data set were used. The deep U-shaped curves for normal samples (blue) and a subset of cancer samples (orange) indicated the promoter was protected from methylation. In 6 cancer samples (red), methylation of the promoter was significantly elevated, indicating loss of protection. FIG. 3E depicts DNA methylation patterns at P16 promoter-flanking region in a pair of matched normal and cancer samples from patient BRD3170. FIG. 3G depicts location of MB-41 matches in the protected region. Each bar represents a match with the height corresponding to −log₁₀(P) where P is the p-value measuring type-I error of the match reported by the MAST program.

FIGS. 4A-4B depict DNA methylation patterns in paired normal and cancer samples in the WGBS dataset. DNA methylation patterns at P16 promoter-flanking region displayed at the same scale as FIG. 3D. The region was divided into 40 windows (window size=200 bps, step size=100 bps). Mean methylation level in each window was plotted. FIG. 4A depicts data from a pair of matched normal and cancer samples from patient BRD3170, indicating the methylation barrier was compromised in cancer. FIG. 4B depicts data from a pair of matched samples from patient BRD3187 (B), indicating the methylation barrier was intact in cancer. The different patterns between the two patients highlight the heterogeneity of DNA methylation in cancers.

FIGS. 5A-5F show experimental validation of the methylation pattern around the P16 TSS in patient samples and cell lines. FIG. 5A depicts loci in the P16 TSS-flanking region selected for targeted sequencing. A total of eight loci (Alu and site 1 to 7) were sequenced. The location of each locus is displayed relative to the repeats and CpG island. Pyrosequencing was performed on all eight loci (red boxes). Bisulfite TA cloning and sequencing was performed to identify accurate methylation boundary in a specific area (blue rectangle). FIG. 5B shows the methylation of the P16 TSS-flanking region was measured by targeted pyrosequencing of 15 normal colon samples (blue), 10 colon cancer samples with a methylated TSS (orange), and 3 colon cancer samples with a methylated TSS (red). The methylation level of each CpG site was displayed as mean±SD. The coordinates of CpG sites were displayed relative to the TSS. FIG. 5C depicts methylation of the P16 TSS-flanking region was measured by targeted pyrosequencing of 4 pairs of matched normal-cancer samples. FIG. 5D depicts mapping of the boundary of the protected region in the P16 promoter in colon cancer tissues of patients 1 to 4 (corresponding to FIG. 5C) by bisulfite TA cloning and sequencing. The sequencing region is indicated in blue in FIG. 5A. Open circles indicate unmethylated CpG sites, while solid circles methylated ones. Each row represents an individual allele. FIG. 5E depicts DNA methylation patterns in the P16 gene promoter were studied by pyrosequencing in four cell lines (MB435: green, unmethylated; LNCaP: pink, partially methylated; CAMA-1: orange, partially methylated; PC3: blue, hypermethylated). FIG. 5F depicts P16 gene expression in the 4 cell lines was determined by qRT-PCR relative to that of GAPDH (internal control). Data are represented as mean±SD.

FIGS. 6A-6B depict cell lines showing patterns of DNA methylation protection in P16 gene promoter. (A-D) DNA methylation of the P16 promoter region in SKBr3 (FIG. 6A), BT474 (FIG. 6B), HMEC (FIG. 6C) and N-LUNG cells (FIG. 6D) were studied by pyrosequencing. The CpG sites tested were indicated in FIG. 5A. DNA methylation levels were higher in repeats than in promoter. FIG. 6E depicts DNA methylation patterns at the downstream boundary of the P16 promoter in LNCaP and CAMA1 cells by bisulfite TA cloning and sequencing method.

FIGS. 7A-7F depict Mapping of the methylation barrier protecting the P16 promoter and the functional sites. FIG. 7A provides the schematic representation of the reporter constructs for determination of the protective strength of each deletion fragment. P16, fragment from the P16 protected region to be inserted; TK, thymidine kinase promoter; GFP-NeoR, fusion reporter gene between green fluorescent protein and neomycin resistance gene. FIG. 7B depicts the protective strength of deletion fragments. Solid horizontal bars on the left indicate the respective P16 deletion fragments and HS4 core element (positive control) inserted into the construct. TK-control has no insert, serving as the negative control. The location of each P16 deletion fragment is plotted relative to the P16 promoter structure shown on the top. The length of each fragment in bps was indicated in its name, e.g. F-1225 is 1225 bps in length. The protective strength of each deletion fragment was calculated as the survival rate of neomycin resistant colonies after the second round of selection over that of the first round. Data are represented as mean±SD. Survival rates significantly higher than the negative control were marked with an asterisk (p<0.05). FIG. 7C depicts TK promoter activity of 4 different constructs in LNCaP cells was determined by GFPneo expression levels tested by qRT-PCR. GAPDH expression levels were used as internal control for normalization. There was no significant difference between different constructs (p>0.05). Data are represented as mean±SD. FIG. 7D shows inverse correlation between GFPneo expression levels and DNA methylation changes in the negative TK-control (reporter only) and the F-126 construct with long term culture. The left y-axis was relative GFP expression levels by qRT-PCR. The earliest time point (2 months after transfection) was defined as ‘1’ and used as reference for normalization. The right y-axis was TK promoter DNA methylation levels by bisulfite TA cloning and pyrosequencing. Data are shown as mean±standard error (SE). Significant difference between TK-control and F-126 were marked with an asterisk (P<0.05). FIG. 7E shows identification of a core element for DNA methylation protection by scanning mutagenesis. DNA methylation level of each construct during long term culture from 1 month to 8 months (1 Mon to 8 Mon) were plotted. Four scanning mutagenesis constructs are illustrated in FIG. 8 (M1 to M4). Data are shown as mean±SE. FIG. 7F depicts block diagram (upper panel) shows the section in the F-73 segment matched to the MB-41 motif (red line). Detailed alignment (lower panel) shows the MB-41 matched section contains the functional M4 site (red box). +: matched nucleotides.

FIG. 8 depicts design of scanning mutagenesis studies for the 73-bp protective region Four different mutated fragments were designed for scanning mutagenesis to cover the 73-bp fragment. Segments of 12˜19 bp in the 73 bp were replaced by null fragments chosen from fragment F-598 (FIG. 7B) that does not have the protective function. The top row shows the wild type sequence. In the bottom four rows, the sequence of the null fragment at each of the 4 mutation sites was shown in red.

FIGS. 9A-9F depict Characterization of MB-41 and dmPIRs. FIG. 9A and FIG. 9B depict DNA methylation spreading patterns in the TSS-flanking region of the MLH1 gene (FIG. 9A) and the CRABP1 gene (FIG. 9B). Top: structure of the region showing the location of TSS, first exon, CpG island, and repeats. Bottom: Methylation patterns displayed as mean methylation level of CpG sites within each of the 40 sliding windows (window size=200 bps, step size=100 bps). FIG. 9C-9E depict histograms and density curves show distribution of sites matched to the MB-41 motif. Distance to TSS (FIG. 9C), CpG island (FIG. 9D), and repeat (FIG. 9E) were plotted. Data from the 2,252 dmPIRs were used. FIG. 9F depicts fractions of different types of repeats in the entire human reference genome (blue), PIRs (orange), and dmPIRs (gray). Significant enrichments in PIRs or dmPIRs as compared to the whole human genome were marked with asterisks (p<0.05).

FIGS. 10A-10E depict DNA methylation pattern in regions surrounding the promoter of CIMP+ marker genes. DNA methylation data of 5 CIMP+ marker genes were retrieved from the WGBS dataset. The gene structures and DNA methylation levels of the corresponding region were shown respectively. Each region was divided into 40 windows (window size=200 bps, step size=100 bps). Data are represented as mean±SD in each window. FIG. 10A-10D depict DNA methylation patterns indicating loss of protection of promoters in cancer samples as compared to normal samples were observed for genes CACNA1G (FIG. 10A), IGF2 (FIG. 10B), NEUROG1 (FIG. 10C) and RUNX3 (FIG. 10D). The protection was intact in cancer samples for the SOCS1 gene (FIG. 10E).

FIGS. 11A-11C depicts details of an MB-41 Motif. FIG. 11A depicts a sequence logo of the MB-41 motif. FIG. 11 B depicts a sequence logo of the 39-bps motif. The MB-41 motif is highly similar to the 39-bps motif derived by increasing iterations to 1000. FIG. 11C depicts a sequence logo of the 48-bps motif. The MB-41 motif is highly similar to the 48-bps motif derived by using scrambled protected sequences as control (reverse strand).

FIG. 12 depicts the MB-41 motif in the P16 gene, showing the MB-41 motif located inside the 728 bp-long CpG island. The MB-41 motif is positioned 611 bp downstream of the CpG island start site and 76 bp upstream of the CpG island end site.

FIGS. 13A-13J depicts transition of methylation level in regions tagged by the 189 dmPIRs. For each tagged region, the repeat and promoter were each divided into five equal-sized sliding windows, and the segment in between was divided into 10 sliding windows (step size=% window size). The Mean±SD of methylation in each window is plotted. Blue line: normal samples. Red line: ACF samples.

FIG. 14A-14B depict motif alignments. FIG. 14A depicts a sequence logo of the 26-bp motif. FIG. 14B depicts alignment of the 26-bps motif derived from esophageal squamous cell carcinoma to the MB-41 motif (reverse strand), showing the high degree of similarity.

The drawing figures do not limit the present inventive concept to the specific aspects disclosed and described herein. The drawings are not necessarily to scale, emphasis instead being placed on clearly illustrating principles of certain aspects of the present inventive concept.

### DETAILED DESCRIPTION

The following detailed description references the accompanying drawings that illustrate various aspects of the present disclosure. The drawings and description are intended to describe aspects and aspects of the present inventive concept in sufficient detail to enable those skilled in the art to practice the present inventive concept. Other components can be utilized and changes can be made without departing from the scope of the present inventive concept. The following description is, therefore, not to be taken in a limiting sense. The scope of the present inventive concept is defined only by the appended claims, along with the full scope of equivalents to which such claims are entitled.

The present disclosure relates to genome screening and functional validation of methylation barriers. Through integrated computational and experimental analysis of methylomes of colorectal cancer and esophageal cancer, more than 600 methylation barriers are discovered that shared a common 41-bp sequence motif (MB-41). Comprehensive in vitro assays validate the protective function of a genetic element carrying the MB-41 motif, which is immediately upstream of the promoter of P16 tumor suppressor gene. Functional sites are fine-mapped and reveal pervasive existence of cis-acting methylation barriers in the human genome that protect promoters and elicited the sequence signature of these barriers. Specific focus is on promoter-associated CGIs juxtaposed with genomic repetitive elements. Repetitive elements are widespread in the human genome and largely silenced via constitutive hypermethylation. It has been proposed that repeats may serve as de novo methylation center and expose adjacent regions to methylation pressure. Promoter-associated CGIs that are near these repeats but remain unmethylated mark promising areas to search for methylation barriers. To enrich for functional barriers, the scan to areas are further limit where normal methylation boundaries are compromised in disease conditions such that promoter-associated CGIs become aberrantly methylated. Colorectal and esophageal cancers, given frequent gene-specific promoter methylation and dysregulated transcription are used in the present study. Colorectal cancer refers to cancer starting either in the colon or the rectum. These cancers can also be called colon cancer or rectal cancer, depending on where they start. Colorectal cancer is a growth of cells that forms in the lower end of the digestive tract. Most of these cancers start as noncancerous growths called polyps. Removing polyps can prevent cancer, so health care providers recommend screenings for those at high risk or over the age of 45. Colonic adenocarcinoma is a type of colorectal cancer that starts in the gland cells that make mucus to lubricate and protect the inside of the colon and rectum. Symptoms may vary depending on the colorectal cancer's size and location. Symptoms might include blood in the stool, abdominal discomfort, and a change in bowel habits, such as diarrhea or constipation. Colorectal cancer treatment depends on the size, location, and how far the cancer has spread. Common treatments include surgery to remove the cancer, chemotherapy, and radiation therapy. Esophageal cancer refers to malignant (cancer) cells formed in the tissues of the esophagus. Squamous cell carcinoma is a common type of esophageal cancer that forms in the thin, flat cells lining the inside of the esophagus. Smoking, heavy alcohol use, and Barrett esophagus can increase the risk of esophageal cancer. Signs and symptoms of esophageal cancer are weight loss and painful or difficult swallowing.

### I. Terminology

The phraseology and terminology employed herein are for the purpose of description and should not be regarded as limiting. For example, the use of a singular term, such as, “a” is not intended as limiting of the number of items. Also, the use of relational terms such as, but not limited to, “top,” “bottom,” “left,” “right,” “upper,” “lower,” “down,” “up,” and “side,” are used in the description for clarity in specific reference to the figures and are not intended to limit the scope of the present inventive concept or the appended claims.

Further, as the present inventive concept is susceptible to aspects of many different forms, it is intended that the present disclosure be considered as an example of the principles of the present inventive concept and not intended to limit the present inventive concept to the specific aspects shown and described. Any one of the features of the present inventive concept may be used separately or in combination with any other feature. References to the terms “embodiment,” “aspects,” and/or the like in the description mean that the feature and/or features being referred to are included in, at least, one aspect of the description. Separate references to the terms “embodiment,” “aspects,” and/or the like in the description do not necessarily refer to the same embodiment and are also not mutually exclusive unless so stated and/or except as will be readily apparent to those skilled in the art from the description. For example, a feature, structure, process, step, action, or the like described in one embodiment may also be included in other aspects but is not necessarily included. Thus, the present inventive concept may include a variety of combinations and/or integrations of the aspects described herein. Additionally, all aspects of the present disclosure, as described herein, are not essential for its practice. Likewise, other systems, kits, methods, features, and advantages of the present inventive concept will be, or become, apparent to one with skill in the art upon examination of the figures and the description. It is intended that all such additional systems, kits, methods, features, and advantages be included within this description, be within the scope of the present inventive concept, and be encompassed by the claims.

Any term of degree such as, but not limited to, “substantially” as used in the description and the appended claims, should be understood to include an exact, or a similar, but not exact configuration. For example, “a substantially planar surface” means having an exact planar surface or a similar, but not exact planar surface. Similarly, the terms “about” or “approximately,” as used in the description and the appended claims, should be understood to include the recited values or a value that is three times greater or one third of the recited values. For example, about 3 mm includes all values from 1 mm to 9 mm, and approximately 50 degrees includes all values from 16.6 degrees to 150 degrees. For example, they can refer to less than or equal to ±5%, such as less than or equal to ±2%, such as less than or equal to 1%, such as less than or equal to ±0.5%, such as less than or equal to ±0.2%, such as less than or equal to ±0.1%, such as less than or equal to ±0.05%.

The terms “comprising,” “including” and “having” are used interchangeably in this disclosure. The terms “comprising,” “including” and “having” mean to include, but not necessarily be limited to the things so described. The term “consisting of” limits membership to the specified materials or item. The term “consisting essentially of” is more limiting than “comprising” but not as restrictive as “consisting of.” Specifically, the term “consisting essentially of” limits membership to the specified materials or items and those that do not materially affect the essential characteristics of the present disclosure.

The terms “or” and “and/or,” as used herein, are to be interpreted as inclusive or meaning any one or any combination. Therefore, “A, B or C” or “A, B and/or C” mean any of the following: “A,” “B” or “C”; “A and B”; “A and C”; “B and C”; “A, B and C.” An exception to this definition will occur only when a combination of elements, functions, steps or acts are in some way inherently mutually exclusive.

As used herein, the term “gene” means a DNA sequence that encodes all the information for the regulated biosynthesis of an RNA product, including promoters, exons, introns, and other untranslated regions that control expression. As used herein, “expression” or “gene expression” includes but is not limited to one or more of the following: transcription of a gene into precursor mRNA; splicing and other processing of the precursor mRNA to produce mature mRNA; mRNA stability; translation of the mature mRNA into protein (including codon usage and tRNA availability); and glycosylation and/or other modifications of the translation product, if required for proper expression and function. Gene expression can be detected by quantitative PCR (qPCR) technique. It monitors the amplification of a targeted DNA molecule during the PCR (i.e., in real time), not at its end, as in conventional PCR. Real-time PCR can be used quantitatively and semi-quantitatively (i.e., above/below a certain amount of DNA molecules). Gene expression can also be observed using a microarray of polynucleotides, an ELISA technique, or a Southern blotting method. As used herein, RT qPCT means Reverse transcription quantitative polymerase chain reaction, which is used to measure a gene expression level. The terms “CpG island” and “CGI” are used interchangeably herein to refer to a region of DNA in a vertebrate genome, which contains a relatively large number of CpG dinucleotide repeats. In mammalian genomes, CpG islands extend for more than 200 bp, up to about 45000 base pairs. CpG islands may appear in the coding strand as well as the reverse complementary strand. A relatively large number of CpG dinucleotide repeats is for example identified when the genomic region has a GC content higher than 50%, and an observed ratio of Cytosine-phosphate-Guanine (CpG) versus expected CpG greater or equal to 0.6 (Gardiner-Garden and Frommer, 1987). A “promoter-associated CpG island” refers to such an island that is within or near a promoter region. The human genome contains about 30,000 CGIs, 62.9% of which are located within ±2 kbps of a TSS.

CpG islands (CGIs) have evolved from a peculiar sequence overrepresentation of CpGs to being recognized as functionally important parts of the genome that define and regulate promoter regions of vertebrates. CGIs near gene promoters are usually associated with lack of DNA methylation and can be considered as the best predictors for defining active or potentially active promoter regions. Methylated CGIs play a role in X-inactivation, genomic imprinting, aberrant methylation patterns in cancer, and gene silencing during cell differentiation. Most importantly, it is believed that CGIs play an important role in fine-tuned regulatory processes by directing gene expression patterns and cell fate, thereby acting as vital landmarks of the epigenome. Protein p16 is a tumor suppressor protein that is a cyclin-dependent kinase inhibitor and is essential in regulating the cell cycle. Protein p16 inactivates cyclin-dependent kinases that phosphorylate Rb; therefore, p16 can decelerate the cell cycle. Rb phosphorylation status in turn influences expression of p16. In one aspect, p16 hypermethylation, mutation, or deletion may lead to downregulation of the gene and can lead to cancer through the dysregulation of cell cycle progression. It should be understood that a CpG island is associated with the nucleotide sequence of the P16 tumor suppressor sequence, for example with SEQ ID NO: 2, in the sense that the P16 sequence encompasses a CpG island. Further, in the P16 gene, the MB-41 motif is located inside the CpG island, which is 728 bp long or about 728 bp long. The MB-41 motif is positioned 611 bp downstream of the CpG island start site, and 76 bp upstream of the CpG island end site. CpG islands may also be found in other tumor suppressor genes, such as p53, p21, Mdm2, PTEN, p14arf, or MDM4.

In any of the disclosed methods, systems, or kits, a “subject” refers to a human, a livestock animal, a companion animal, a lab animal, or a zoological animal. Non-limiting examples of a subject are a rodent, e.g., a mouse, rat, or guinea pig, etc.; pig, cow, horse, goat, sheep, llama and alpaca; dog, domestic cat, rabbit, and bird; non-human primate, large cat, wolf, and bear.

### 1. Whole and Partial Genome Sequencing

The methods, systems, or kits of the present disclosure utilize whole and/or partial genome sequencing. Such methods and systems or kits are able to achieve whole or partial genome sequencing through either microarray-based sequence genotyping studies or whole genome sequencing. Two approaches are available for assembling short shotgun sequence reads into longer contiguous genomic sequences. In the de novo assembly approach, sequence reads are compared to each other, and then overlapped to build longer contiguous sequences. Alternatively, the reference-based assembly approach involves mapping each read to a reference genome sequence. In any approach, genome sequencing is well understood to be able to identify genetic variation (single nucleotide polymorphisms, small indels, and copy number variants), build haplotypes from genome assemblies, identify polymorphisms in samples comprising mixtures of genomes, and determine and/or monitor the polymorphisms. Unless otherwise indicated, the practice of the present invention involves conventional techniques commonly used in molecular biology, microbiology, protein purification, protein engineering, protein and DNA sequencing.

### II. Determination of Gene Methylation

The methods, systems, or kits of the present disclosure comprise measuring the level of methylation of a combination of certain genetic elements such as CpG islands in a genomic sample obtained for example from a biological sample obtained from the subject. A biological sample may include, but is not limited to, a cell, a cellular organelle, an organ, a tissue, a tissue extract, a biofluid, or an entire organism. The sample may be a heterogeneous or homogeneous population of cells or tissues. As such, methylation levels of selected genetic elements such as CpG islands can be measured within cells, tissues, organs, or other biological samples obtained from the subject. For instance, the biological sample can be bone marrow extract, whole blood, blood plasma, serum, peripheral blood, urine, phlegm, synovial fluid, milk, saliva, mucus, sputum, exudates, cerebrospinal fluid, intestinal fluid, cell suspensions, tissue digests, tumor cell containing cell suspensions, cell suspensions, and cell culture fluid which may or may not contain additional substances (e.g., anticoagulants to prevent clotting). In some aspects, multiple biological samples may be obtained for diagnosis by the methods of the present invention, e.g., at the same or different times. A sample, or samples obtained at the same or different times, can be stored and/or analyzed by different methods.

### III. Genome Computational Analysis

The methods, systems, or kits of the present disclosure utilize whole genome sequencing assay, with a focus on methylation sequencing assay. The sequence reads of the sequencing assays are processed through corresponding computational analyses, also hereafter referred to any one of computational pipelines, computational assessments, and computational analyses. Each computational analysis identifies values of features of sequence reads that are informative for generating a cancer prediction while accounting for interfering signals (e.g., noise). As an example, small variant features (e.g., features derived from sequence reads that were generated by a small variant sequencing assay) can include a total number of somatic variants. As another example, whole genome features (e.g., features derived from sequence reads that were generated by a whole genome sequencing assay) can include a total number of copy number aberrations. As yet another example, methylation features (e.g., features derived from sequence reads that were generated by a methylation sequencing assay) can include a total number hypermethylated or hypomethylated regions. Additional features that are not derived from sequencing-based approaches, such as baseline features that can refer to clinical symptoms and patient information, can be further generated and analyzed. In some aspects, one, two, three, or all four of the types of features (e.g., small variant features, whole genome features, methylation features, and baseline features) can be provided to a single predictive cancer model that generates a cancer prediction. In some aspects, the values of different types of features can be separately provided into different predictive models. Each separate predictive model can output a score that then serves as input into an overall model that outputs the cancer prediction. Aspects disclosed herein describe a method for detecting the presence of DNA methylation, the method comprising: obtaining sequencing data generated from a plurality of cell-free nucleic acids in a test sample from the subject, wherein the sequencing data comprises a plurality of sequence reads determined from the plurality of cell-free nucleic acids; analyzing, using a suitable programed computer, the plurality of sequence reads to identify two or more sequencing based features; and detecting the presence of cancer based on the analysis of the two or more features.

In certain aspects, sequence reads generated from application of a whole genome sequencing assay are processed using computational analysis, otherwise referred to as a whole genome computational analysis. The computational analysis outputs whole genome features. Sequence reads generated from application of a small variant sequencing assay are processed using a computational analysis, otherwise referred to as a small variant computational analysis. The computational analysis outputs small variant feature(s). Sequence reads generated from application of a methylation sequencing assay are processed using computational analysis, otherwise referred to as a methylation computational analysis. The computational analysis outputs methylation features.

### IV. Systems and Kits

The present disclosure provides methods, systems, or kits for detecting the presence of a methylation barrier in a genomic sample of a subject, and the risk of or presence of disease such as a cancer in the subject. In some aspects, the system or kit herein can include a unit for sample collection, a unit for sample treatment, a unit or processor for genome analysis, and instructions for use of any of the foregoing in accordance with any of the methods described herein. The system or kit may further include a description of selecting an individual suitable for treatment based on identifying whether that individual has the target disease, e.g., applying the diagnostic method as described herein. In still other aspects, the instructions can include a description of administering a therapeutic active agent to an individual at risk of the target disease. In one aspect, the system or kit comprises (a) a biological sample comprising a genomic DNA sequence; (b) a reagent selected from one or more from the group consisting of a DNA extraction reagent, a bisulfite treatment reagent, a primer, a PCR reagent, a reagent for next-generation sequence, and a reagent to measure methylation level in the biological sample; (c) a control sample comprising LNCaP cell line DNA and a P16 gene primer; and (d) instructions for a user. In one aspect, the user instructions comprise the steps of (i) processing the biological sample to a bisulfite conversion process and a sequencing preparation step; (ii) comparing the processed biological sample with the control sample to ensure proper implementation of bisulfite conversion and sequencing preparation; (iii) obtaining genomic DNA sequence information from the processed biological sample, wherein the genomic DNA sequence information comprises a sequence of at least one transcription start site (TSS); (iv) searching a region within the TSS and about ±200 bps flanking the TSS region for each occurrence of a combination of a promoter sequence, a CpG island, and a repeat sequence, wherein the combination is a promoter-CpG island (island)-repeat (PIR) trio indicative of methylation barrier; (v) determining methylation of each occurrence of the combination of the promoter sequence, the CpG island and the repeat sequence against that of the control sample; and (vi) identifying the presence of the cancer or increased risk of the cancer in the subject when the subject possesses an increased methylation or a compromised methylation barrier comparing with that of the control sample. In one aspect, the system or kit comprises (a) a first database configured to store information regarding a genome of the subject obtained by whole or partial genome sequencing, wherein the information comprises a sequence of at least one transcription start site (TSS); (b) a processor configured to perform (i) instructions for searching a region including the TSS and about ±2000 bps flanking the TSS region for each occurrence of a combination of a promoter sequence, a CpG island, and a repeat sequence, wherein the combination is a promoter-CpG island (island)-repeat (PIR) trio indicative of a candidate methylation barrier; (ii) instructions to identify dmPIRs by determining the relative methylation of any one of, or any combination of the promoter, the CpG island and the repeat of the PIR trio compared to the methylation the one of, or the combination of the promoter, the CpG island and the repeat of the PIR trio obtained from a normal control sample; (iii) instructions for locating dmPIR-tagged regions by aggregating dmPIRs around the same TSS; and (iv) instructions for determining methylation spreading in dmPIR-tagged regions, wherein increased methylation of the one of, or the combination of any of the promoter, the CpG island and the repeat of the PIR trio compared to the normal control sample is indicative of the presence of a compromised methylation barrier in the genome of the subject and indicative of the presence of a cancer or increased risk of cancer in the subject. In another aspect, the presence of the compromised methylation barrier is indicative of a colorectal cancer or an esophageal cancer in the subject, and the presence of the compromised methylation barrier is indicative of a colonic adenocarcinoma or an esophageal squamous cell carcinoma in the subject. In some aspect, the at least one CpG island is promoter-associated. In yet another aspect, the compromised methylation barrier comprises a nucleotide sequence of SEQ ID NO: 1 [41-bp motif (MB-41)]. In one aspect, the “B” in SEQ ID NO: 1 is selected from C, G and T. In another aspect, the “S” in SEQ ID NO: 1 is selected from C and G. In another aspect, the “K” in SEQ ID NO: 1 is selected from T and G. In another aspect, the “Y” in SEQ ID NO: 1 is selected from C and T. In some aspect, the at least one CpG island is associated with the nucleotide sequence of the P16 tumor suppressor having the sequence of SEQ ID NO: 2. In another aspect, the at least one CpG island is associated with the nucleotide sequence of another tumor suppressor gene.

The system or kit may optionally provide additional components such as sample container and interactive interface. Further, the sample container may have a label or package insert(s) on or associated with the container. In one aspect, the system or kit comprises (a) a biological sample wherein a genomic DNA sequence can be obtained; (b) a reagent selected from one or more from the group consisting of a DNA extraction reagent, a bisulfite treatment reagent, a primer, a PCR reagent, a reagent for next-generation sequence, and a reagent to measure methylation level in the biological sample; (c) a control sample comprising LNCaP cell line DNA and a P16 gene primer; (d) instructions for a user. Such instructions comprise the steps of: (i) processing the biological sample to a bisulfite conversion process and a sequencing preparation step; (ii) comparing the processed biological sample with the control sample to ensure proper implementation of bisulfite conversion and sequencing preparation; (iii) obtaining the genomic DNA sequence information from the processed biological sample, wherein the genomic DNA sequence information comprises a sequence of at least one transcription start site (TSS); (iv) searching a region within the TSS and about ±200 bps flanking the TSS region for each occurrence of a combination of a promoter sequence, a CpG island, and a repeat sequence, wherein the combination is a promoter-CpG island (island)-repeat (PIR) trio indicative of methylation barrier; (v) determining methylation of each occurrence of the combination of the promoter sequence, the CpG island and the repeat sequence against that of the control sample; and (vi) identifying the presence of the cancer or increased risk of the cancer in the subject when the subject possesses an increased methylation or a compromised methylation barrier comparing with that of the control sample. In some aspects, the present disclosure provides articles of manufacture comprising contents of the system or kit described above.

Having described several aspects, it will be recognized by those skilled in the art that various modifications, alternative constructions, and equivalents may be used without departing from the spirit of the present inventive concept. Additionally, a number of well-known processes and elements have not been described in order to avoid unnecessarily obscuring the present inventive concept. Accordingly, this description should not be taken as limiting the scope of the present inventive concept.

Those skilled in the art will appreciate that the presently disclosed aspects teach by way of example and not by limitation. Therefore, the matter contained in this description or shown in the accompanying drawings should be interpreted as illustrative and not in a limiting sense. The following claims are intended to cover all generic and specific features described herein, as well as all statements of the scope of the method and assemblies, which, as a matter of language, might be said to fall there between.

### EXAMPLES

The following examples are included to demonstrate preferred aspects of the disclosure. It should be appreciated by those of skill in the art that the techniques disclosed in the examples that follow represent techniques discovered by the inventor to function well in the practice of the present disclosure, and thus can be considered to constitute preferred modes for its practice. However, those of skill in the art should, in light of the present disclosure, appreciate that many changes can be made in the specific aspects which are disclosed and still obtain a like or similar result without departing from the spirit and scope of the present disclosure.

### Example 1. Study Design

The present study was aimed to perform genome-wide scanning of cis-acting methylation barriers that protect gene promoters, construct the sequence signature, and validate the protective function experimentally. Promoter-associated CGIs juxtaposed with genomic repetitive elements were a specific focus. Repetitive elements are widespread in the human genome and largely silenced via constitutive hypermethylation. It has been proposed that repeats may serve as de novo methylation center and expose adjacent regions to methylation pressure. Therefore, promoter-associated CGIs that are near these repeats but remain unmethylated mark promising areas to search for methylation barriers. To enrich the field with likely functional barriers, the scan was further limited to areas where normal methylation boundaries are compromised in disease conditions such that promoter-associated CGIs become aberrantly methylated. Colorectal cancers, given frequent gene-specific promoter methylation and dysregulated transcription were used as a model for this purpose. The present study reported an integrated computational and experimental investigation on methylomes of colorectal cancer.

### Example 2. Materials and Procedure

Discovery data set. Published methylome data published by was used to identify dmPIRs and non-dmPIRs and to discover sequence motifs. (Johnstone, S. E., Reyes, A., Qi, Y., Adriaens, C., Hegazi, E., Pelka, K., Chen, J. H., Zou, L. S., Drier, Y., Hecht, V., et al. (2020). Large-Scale Topological Changes Restrain Malignant Progression in Colorectal Cancer. Cell 182, 1474-1489 e1423. 10.1016/j.cell.2020.07.030). This data set reported Beta-values at single base pair resolution produced by whole-genome bisulfite sequencing (WGBS). It included 3 pairs of matched normal and cancer samples and 23 individual cancer samples of colonic adenocarcinomas. This data set was downloaded from the NCBI GEO database (GSE133928).

Replication data sets. Two independent data sets were compiled to examine the replicability of dmPIRs and MB-41 motif. The first data set contained 10 pairs of matched normal and cancer samples of colorectal cancers. Methylation levels were reported as Beta-value at single base pair resolution from reduced representation bisulfite sequencing (RRBS). It also contained RNA-seq data that reported TPM values for each gene transcript in these samples. this data set was downloaded from the GEO database (methylome: GSE95656, transcriptome: GSE95132). The second data set contained methylation profiles of 11 normal gastrointestinal tissues that reported Beta-values produced by WGBS. This data set included 2 normal colon samples from different studies and was downloaded from the GEO database (GSE32399 and GSE52271), and 4 small intestine samples and 5 large intestine samples from the ENCODE project sample IDs: ENCFF286MDT, ENCSR113KSF, ENCSR265ZHH, ENCSR357BWB, ENCFF509ORM, ENCFF002HXC, ENCFF911ZIV, ENCSR987GWQ, and ENCSR331PWE.

The discovery data set and the two replication data sets reported position-specific methylation values based on different versions of the human reference genome. Using the Liftover program, these data was mapped to the hg19 version. All coordinates reported in the present study were based on the hg19 version.

Cell lines and tissues for in vitro studies. Cell lines were obtained from the American Type Culture Collection (Manassas, VA). All normal and tumor tissue of patient samples were obtained from the tissue bank at the University of Texas M. D. Anderson Cancer Center (Houston, TX) and the Johns Hopkins Hospital (Baltimore, MD). Patients gave informed consent for the collection of residual tissue as per institutional guidelines. The studies were approved by the institutional review board of the M. D. Anderson Cancer Center.

### Example 3. Computational Methods and Data Treatments

Identification of PIR trios: Given a TSS, a PIR is a set of three DNA fragments consisting of a promoter (i.e., ±200 bps flanking the TSS), a CGI, and a repetitive element, all of which are within the ±2 kbps TSS-flanking region. To identify these trios, the UCSC Genome Browser annotations were queried on the GRCh37/hg19 human reference genome. If multiple repeats and/or CGIs were found within the ±2 kbps flanking a TSS, each unique combination was considered as a trio. CGIs and repeats extending outside the ±2 kbps TSS-flanking region were truncated.

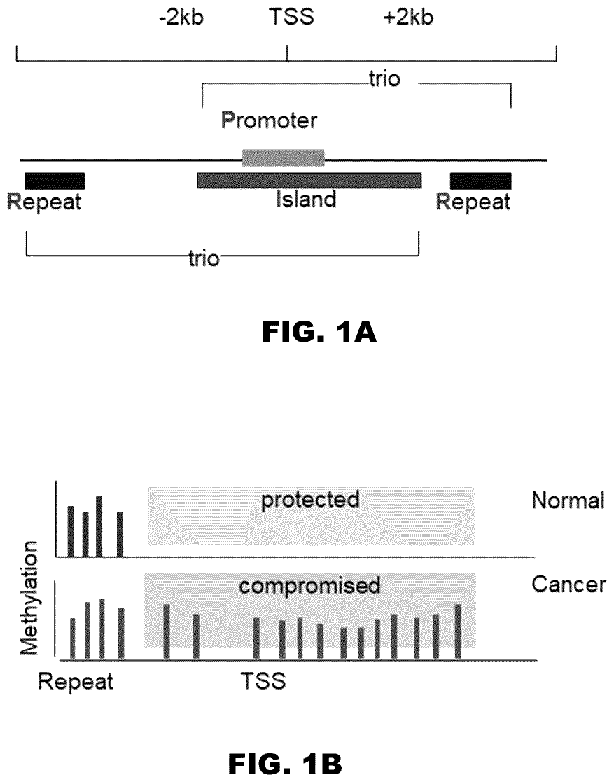

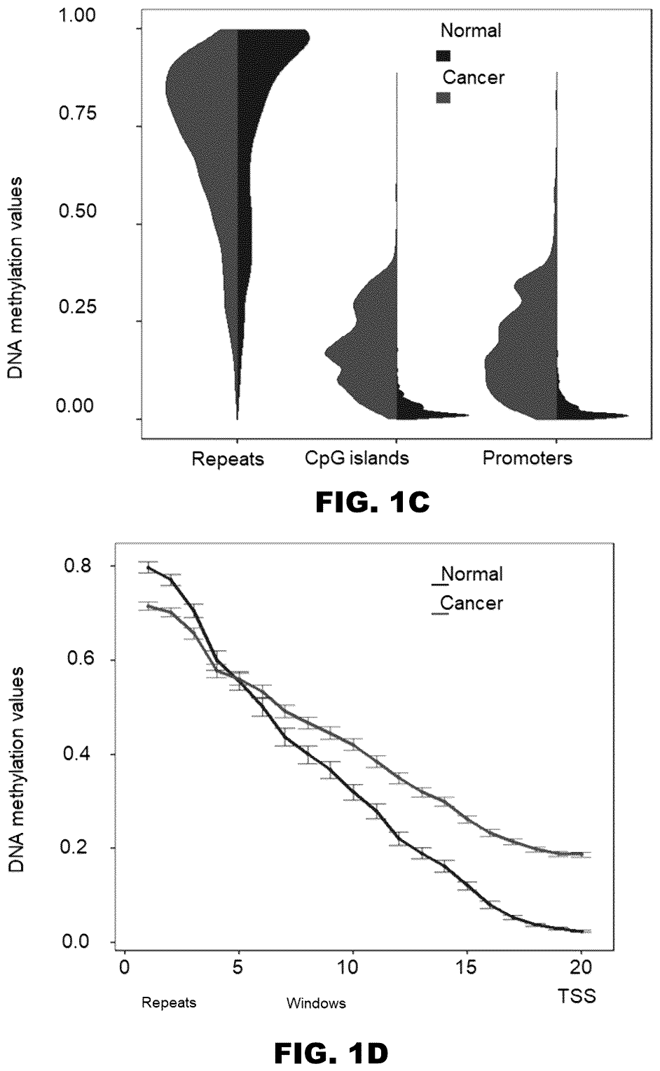

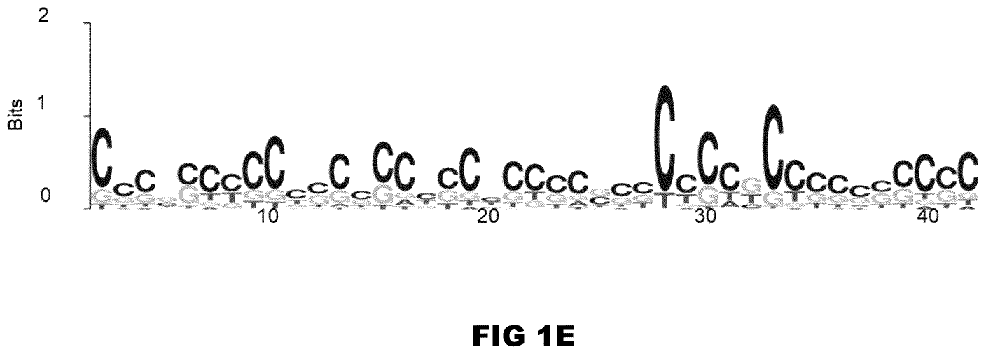

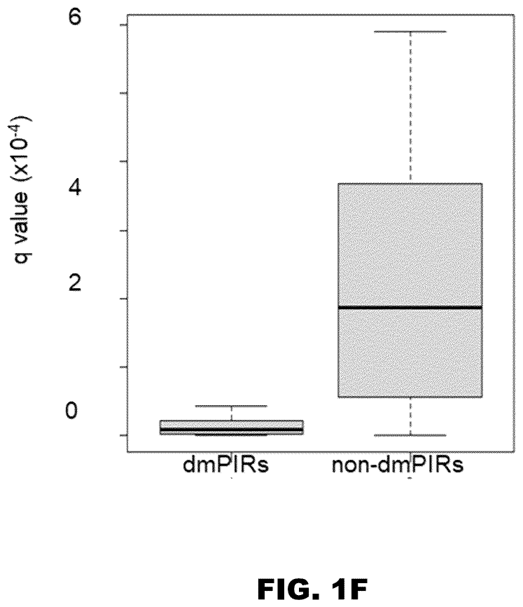

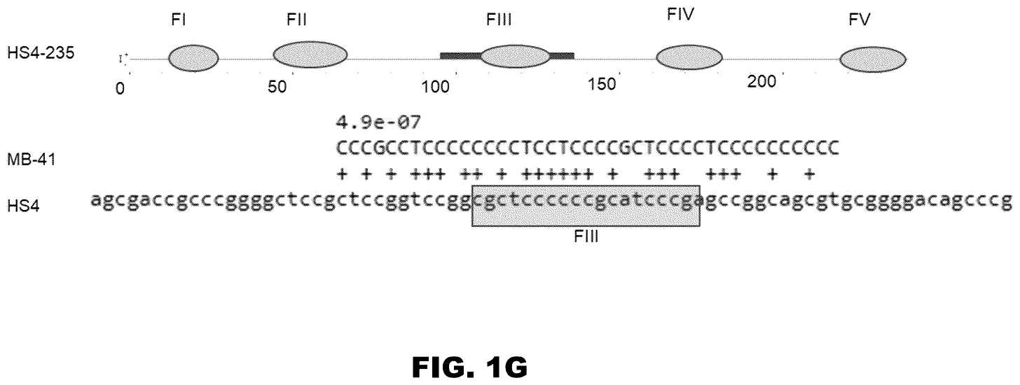

Discovery of dmPIRs and non-dmPIRs. Methylation data were downloaded as Beta-values representing the percentage of methylated reads over total reads covering a specific base pair. Beta-value was converted to M-value via logit transformation to reduce heteroscedasticity and used M-value in statistical analyses. Given a genomic position g, DNA methylation level in a sample was denoted as M_{g}^{s}. For each PIR trio, we built a linear mixed-effects model (LMM) to test if the methylation level of a position was associated with its location L_{g}∈{promoter, repeat, CGI} and the clinical diagnosis Dₛ∈{Normal, Cancer} of the sample

$${M}_{g}^{s} \sim {L}_{g} + {D}_{s} + {L}_{g} : {D}_{s} + ( 1 \vert Patient ) \qquad [ 1 ]$$

where L_{g}, Dₛ and their interactions L_{g}:Dₛ were defined as fixed effects and patients who contributed paired normal-cancer samples were defined as random effects. To correct for multiple comparison, nominal p-values were adjusted to false discovery rates (FDRs) using the Benjamini-Hochberg method. an FDR<0.1 threshold was applied to detect significant main effects and interactions. Post hoc pairwise comparisons were then performed using the Tukey-HSD method and applied a series of filters (Table 1) to identify PIRs that matched the methylation patterns in FIG. 1A. We used a P value cutoff of 0.05 for post hoc pairwise comparisons and further required a fold change >4. PIRs passing these filters were differentially methylated (dmPIRs), indicating existence of methylation barriers that were functional in normal samples but compromised in cancer samples. Conversely, non-dmPIRs were trios that had p value >0.1 for main effects, p value >0.1 for interaction, and mean methylation level >70% in all groups. Regions marked by non-dmPIRs likely did not contain methylation barriers.

TABLE 1
Filters used in post-hoc pairwise comparisons

| Filter | Interpretation |
|---|---|
| E(M̅\|L = P, D = N) < E(M̅\|L = R, D = N) | In normal samples, the promoter and the CGI in a PIR |
| E(M̅\|L = I, D = N) < E(M̅\|L = R, D = N) | were hypomethylated as compared to the repeat. |
| E(M̅\|L = P, D = C) > E(M̅\|L = P, D = N) | The promoter and the CGI in a PIR were |
| E(M̅\|L = I, D = C) > E(M̅\|L = I, D = N) | hypermethylated in cancer samples as compared to |
| | normal samples. |
| E(M̅\|L = P, D = C) ≤ E(M̅\|L = R, D = C) | In cancer samples, the methylation level of the |
| E(M̅\|L = I, D = C) ≤ E(M̅\|L = R, D = C) | promoter and the CGI in a PIR was not higher than |
| | that of the repeat. |

Discovery and analysis of sequence motifs. Methylation boundary was identified in each dmPIR region by finding the longest segment that were unmethylated in normal samples. Given a dmPIR, the ±2000 bps TSS-flanking region was divided into 40 sliding windows (window size=200 bps, step size=100 bps). Windows with mean Beta-value <0.1 were considered unmethylated. The coordinates of the most upstream and the most downstream unmethylated window marked the methylation boundaries. If multiple dmPIRs had overlapping protected segments, their methylation boundaries were merged by finding the most upstream and most downstream coordinates. The collection of DNA sequences inside these boundaries represented regions protected from methylation, which were hypothesized sharing common sequence motifs.

the MEME program (version 5.4.1) was used to discover motifs that were enriched in protected sequences as compared to unprotected sequences. The MEME default parameters were used: motif length range 6-50 bps, total occurrence range 2-600, searching both strands (coding strand and/or the reverse complementary strand), allowing multiple non-overlapping occurrences of a motif in a single sequence, no restriction to palindromes, and no sequence shuffling.

For an identified motif, its occurrences were scanned in protected sequences and in unprotected sequences using the FIMO program (p-value <10⁻⁴). To scan the MB-41 motif in the HS4 element, the MAST program was used (p-value <10⁻⁴). Such scanning were done in coding strand and/or the reverse complementary strand. Both DNA strands and filtered motif with E-value <10 were analyzed.

To scan TFBS in the MB-41 motif, the TOMTOM program was used against the Human DNA and HOCOMOCO Human (v11 core) dataset. Similarity was measured by Pearson correlation coefficient. E-value <10.

Replication analysis of dmPIRs and MB-41 motif. For each dmPIR discovered from the WGBS data sets, the same LMM model in equation [1] was used and the same set of filters in Table 1 to test if the patterns could be replicated in the RRBS data set.

The other replication data set contained only normal samples. We therefore modified equation [1] to remove the Dₛ+L_{g}:Dₛ terms. For each dmPIR, a LMM was built

$${M}_{g}^{s} ∼ {L}_{g} + ( 1 \vert Sample ) \qquad [ 2 ]$$

where location had fixed effects and individual samples had random effects. At FDR<0.1, dmPIRs that had significant higher methylation level in repeats than in promoters and CGIs were considered replicable.

Association analysis between methylation and gene expression. Samples collected by Rosenberg et al. had RRBS methylome data and RNA-seq transcriptome data. The dmPIRs were mapped to gene transcripts based on UCSC Genome Browser annotations. The association between DNA methylation level of dmPIRs were tested and gene expression was studied in RRBS data in 9 colon cancer sample pairs. The association between fold change of DNA methylation on the ENSTs promoter region (TSS±200 bp) and relative expression levels were analyzed with Pearson's correlation. Differentially expressed genes were retrieved from the original RRBS study that analyzed RNA-seq data using the DESeq2 package.

Gene annotation and gene set enrichment analysis. UCSC genome browser (GRCh37/hg19 human genome) database was used to find genes encoded in a genomic region. For gene set enrichment analysis, the Panther program was used that performed Fisher's test to compare the query genes list with all genes in the human genome, and conduct Benjamini-Hochberg correction for multiple comparisons. FDR <0.1 indicated significant enrichment. For cancer related gene analysis, GUST database was used. Probability of tumor suppressor >0.5 was used as cut off for general tumor suppressor classification. Probability of tumor suppressor >0.95 was used as cut off for high confident classification of tumor suppressor.

### In Vitro Experimental Methods

DNA bisulfite treatment. Genomic DNA was extracted from patient samples and cell line samples and treated with bisulfite following standard procedures such as described in the art, for example in J. Shu et al., Silencing of bidirectional promoters by DNA methylation in tumorigenesis, Cancer Res 66, 5077-5084 (2006). Samples of 2 μg genomic DNA were treated by 0.2 M NaOH at 37° C. for 10 minutes, and then incubated with 30 μl 10 mM hydroquinone and 520 μl 3M sodium bisulfite at 50° C. for 16 hours. DNA was purified with a Wizard miniprep Column (Promega, Madison, WI), and precipitated with ammonium acetate and ethanol method.

Bisulfite-Pyrosequencing and TA cloning and sequencing. Bisulfite-pyrosequencing was carried out as previously described. Two rounds of PCR were used to synthesize biotin-labeled specified PCR products. Primer sequences are listed in Table 2. The PCR products were captured by Streptavidin Sepharose HP beads (Amersham Biosciences, Uppsala, Sweden) and denatured with a Pyrosequencing Vacuum Prep Tool (Qiagen, Valencia, CA). Pyrosequencing primers (0.3 μM) were annealed to the single stranded PCR products and pyrosequencing was performed using the PSQ HS 96 Pyrosequencing System. Quantification of cytosine methylation was performed using the provided software (PSQ HS96A 1.2, available from Qiagen, Valencia, CA). Pyrosequencing results were confirmed by TA cloning and sequencing in selected samples. Bisulfite-PCR products were cloned into a pCR 4.0-TOPO vector (Invitrogen, Carlsbad, CA) and 8˜12 clones were selected and sequenced at the DNA sequencing core facility at the University of Texas M. D. Anderson Cancer Center. The methylation level was calculated as the average value of the selected clones.

TABLE 2
Primers used in bisulfite pyrosequencing,
TA cloning and sequencing and qRT-PCR.

| | | Pyro- |
|---|---|---|
| | | sequencing |
| Primers | PCR-primers | primers |
| p16-1 | F: 5′ GTATTTTTTTTGGTTTAGGAATTATG 3′ | 5′ CCCTTCCC |
| | RU: 5′ BioUACCCTAATTCAAAAAATTCCTTTTAA3′ | CTCCTAC 3′ |
| | R: 5′ ACCCTAATTCAAAAAATTCCTTTTAA 3′ | |
| p16-2 | F: 5′ TTTGGTAGTTAGGAAGGTTGT 3′ | 5′ TGGTAGTT |
| | RU: 5′ BioUACCTCCCTACTCCCAACC 3′ | AGGAAGGTTGT |
| | R: 5′ ACCTCCCTACTCCCAACC 3′ | A 3′ |
| p16-3 | F: 5′ GGTTGTTTTYGGTTGGTGTTTT 3′ | 5′ TTTTTGTT |
| | RU: 5′ BioUACCCTATCCCTCAAATCCTCTAAAA 3′ | TGGAAAGAT |
| | R: 5′ ACCCTATCCCTCAAATCCTCTAAAA 3′ | 3′ |
| p16-4 | F1: 5′ GAGGGGTTGGTTGGTTATTAGA 3′ | 5′ GAGGGGGA |
| | RU: 5′ BioUGAGAGAGTTTAGTTTTTGGATTAG3′ | GAGTAGGTA |
| | R1: 5′CCTATACACCCTATATTACCTACTAATAA3′ | 3′ |
| | F2: 5′ AGGGGTTGGTTGGTTATTAG 3′ | |
| p16-5 | F: 5′ GTTTGTAGGGGAATTGGA 3′ | 5′ GGGAATTG |
| | RU: 5′ BioUCCTCATTCCTCTTCCTTAACT 3′ | GAATTAGGTA |
| | R: 5′ CCTCATTCCTCTTCCTTAACT 3′ | 3′ |
| p16-6 | F: 5′ GGGGAATATATTTGTATTAGATGG 3′ | 5′ CCCTTTTT |
| | RU: 5′ BioUCCCAACACATCTTACATTTCTT 3′ | ATCCCAAAC |
| | R: 5′ CCCAACACATCTTACATTTCTT 3′ | 3′ |
| p16-7 | F: 5′ TGTGGTGTATGTTGGAATAAAT 3′ | 5′ AATTACAA |
| | RU: 5′ BioUTCTCCCAAAATAAAAAAATTACAA 3′ | AACRTAAAACA |
| | R: 5′ TCTCCCAAAAAAAAAAATTACAA 3′ | C 3′ |
| P16-sine- | F1: 5′ gggtggtggagggtgtttat 3′ | S1:5′ AGGTT |
| up | R1: 5′ cctatctccttcacacttctcaca 3′ | GGAGTGTAATG |
| | F2: 5′ TTTGTTTTTTAGGTTGGAGTGTA 3′ | G 3′ |
| | RU2: 5′ BioUAACATAACCAAACCCTATCTCTACTA 3′ | |
| | R2: 5′ AACATAACCAAACCCTATCTCTACTA 3′ | S2:5′ TTTTT |
| | F3: 5′ TTTTAGTTTTTTGAGTAGTTGGAAT 3′ | GAGTAGTTGGA |
| | RU3: 5′ BioUACACCTATAATCCCAACACTT 3′ | ATTATAT 3′ |
| | R3: 5′ ACACCTATAATCCCAACACTT 3′ | |
| TK-771 | F: 5′ gggtttggttttggtggtta 3′ | |
| | R: 5′ caaacccaatttctattaatctcctt 3′ | |
| TK-1 | F: 5′ ttgtatgtttttagttttatgatga 3′ | 5′ TTTAGTTT |
| | RU: 5′ BioUCACCTTAATATACCAAATAAACCTAAAAC 3′ | AGAGTTTTGTT |
| | R: 5′ CACCTTAATATACCAAATAAACCTAAAAC 3′ | ATTG 3′ |
| TK-2 | F: 5′GGAAGAAATATATTTGTATGTTTTTAGTTTTATGA 3′ | 5′ TTTTAGGT |
| | RU: 5′ BioUCTCACCACCAACTTCTACAACTTAAATT 3′ | TTATTTAGTAT |
| | R: 5′ CTCACCACCAACTTCTACAACTTAAATT 3′ | ATTAAGG 3′ |
| TK-4 | F: 5′ GGATTGTAGGAGTTTTAGGGAGTG 3′ | 5′TAGGAGTTT |
| | RU: 5′ BioUAAACCCTAAACCAAATTTATATCATC 3′ | TAGGGAGTGG |
| | R: 5′ AAACCCTAAACCAAATTTATATCATC 3′ | 3′ |
| BioU | 5′ BiotinGGGACACCGCTGATCGTTTA 3′ | |

| Primers of Real-time PCR for ChIP assays | | Probes |
|---|---|---|
| P16-Site 1 | F: 5′ TTTGAAGCTGGTCTTTGGATCA3′ | 5′ TGTGCAAC |
| | R: 5′ GACCAGAAAAAGTGCTCAGTGTTC 3′ | TCTGCTTC 3′ |
| P16-Site 2 | F: 5′ GGGCGGATTTCTTTTTAACAGA 3′ | 5′ TGAACGCA |
| | R: 5′ CGCCTGCCAGCAAAGG 3′ | CTCAAAC′ |
| P16-Site 3 | F: 5′ CCAACGCACCGAATAGTTACG 3′ | 5′ TCGGAGGC |
| | R: 5′ TTCCAATTCCCCTGCAAACTT 3′ | CGATCC 3′ |
| P16-Site 4 | F: 5′ GATACCTGGATGGAGCTTATCTTTCT 3′ | 5′ ACTAGGAG |
| | R: 5′ TTCCAACATACACCACAGATTTCC 3′ | GGATTATCA |
| | | 3′ |

RNA purification and reverse transcription-CR (RT-PCR). RNA was extracted by standard methods. Gene expression was analyzed by qRT-PCR. Reverse transcription was performed with High-Capacity cDNA kit (Applied Biosystems, Foster City, CA). Taqman real-time PCR was used with Taqman ABI PRISM 7000HT Sequence Detection System (Applied Biosystems) according to the manufacturer's instruction. GAPDH was used for normalization.

Plasmid Construction. The reporter vector for methylation protection pRP-MP was constructed as follows: TK promoter from pRL-TK plasmid (Promega, Madison, WI, USA) and cDNA for GFP and neomycin resistance gene (GFPneo from Dr. Kazuhiro Oka, Baylor College of Medicine, Houston, TX) were ligated together to produce TK-GFPneo reporter cassette, which was inserted into pBluescript-KS vector (Stratagene, La Jolla, CA) to produce pRP-MP plasmid. 11 P16 serial deletion fragments and 4 mutation fragments were PCR amplified (primer sequences listed in Table 3) or synthesized and cloned into pRP-MP both upstream and downstream of the TK-GFPneo cassette to generate the individual reporter plasmids for each fragment to be tested.

TABLE 3
Primer Sequences

| | Site 4 | Site 5 |
|---|---|---|
| ID: N-PRO | 0.7 | 9.5 |
| ID: Jurkat | 2.4 | 10.4 |
| ID: HL-03 | 1.9 | 8.1 |
| ID: HL-06 | 0.0 | 10.4 |
| ID: CEM | 0.0 | 1.8 |
| ID: H69 | 6.5 | 6.8 |
| ID: LNCaP | 4.2 | 44.5 |
| ID: RKO | 92.9 | 95.0 |
| ID: BT474 | 4.4 | 22.0 |
| ID: HMEC | 41.0 | 83.9 |
| ID: SW48 | 91.4 | 97.0 |
| ID: Jurkat | 0.0 | 6.9 |
| ID: MB | 1.0 | 6.6 |
| ID: BJAB | 93.3 | 97.1 |
| ID: TALL | 91.3 | 97.2 |
| ID: SKBr3 | 3.5 | 18.1 |
| ID: RAJI | 93.4 | 100.0 |
| ID: N-LUNG | 2.6 | 13.1 |
| ID: SK | 2.1 | 6.4 |
| ID: CAMA-1 | 4.8 | 65.6 |
| ID: IMR90 | 14.5 | 9.8 |

Methylation protection assay: To measure the protection strength of each serial deletion fragment, the reporter plasmids containing these fragments were linearized by SspI and transfected into LNCaP cells in 6-well plates with lipofectamine 2000 (invitrogen) according the manufacturer's instructions. The cells were transferred into 25 cm² flask 24 hours after transfection and cultured in 600-800 μg/ml Neomycin for 7 days. The number of surviving colonies was recorded. These cells were then grown without neomycin for 2 weeks to allow heterochromatin and methylation spreading, and neomycin selection was resumed for another 8 days. Colonies surviving the 2nd selection were again counted. Survival rate was calculated between control and each serial deletion fragment. Four independent experiments were performed for each construct. Paired sample one-tailed t test was used to test if the survival rate of cells infected with different constructs was higher than that of negative control constructs. A p value <0.05 indicates statistical significance.

To measure the effects of deletion fragments on the rate of DNA methylation spreading, 8 μg control plasmid or the construct with the deletion fragment were linearized by SspI and transfected into LNCaP cell lines in a 6-well plate by lipofectamine 2000 (Invitrogen). The cells were transferred to a 25-cm² flask 24 hours later and cultured in 500-600 μg/ml Neomycin for about 7 days for selection. Surviving cells were pooled and cultured in Neomycin free medium. Cells were collected monthly. DNA methylation level of three CpG sites in the TK promoter were evaluated with pyrosequencing or bisulfite sequencing as described earlier. Three independent experiments were carried out.

### Example 4: Results and Discussion

### Genome-Wide Screening Located Regions Harboring Potential Methylation Barriers

The UCSC Genome Browser annotated 186,296 TSSs, 30,344 CGIs, and 5,481,341 repeats in the hg19 human reference genome. For a given TSS, the ±200 bps flanking region was used as a proxy for the promoter and searched the ±2,000 bps for CGIs and repeats. A promoter, a CGI, and a repeat near the TSS constituted a PIR trio, were used as a tag to mark and examine the area for potential methylation barriers (FIG. 1A). If a TSS had multiple CGIs or multiple repeats within ±2 kbps, each unique combination was a combination of a promoter, CGI (island) and repeat (PIR). A total of 381,180 PIRs were identified, among which 5,577 TSSs had a single PIR, and 65,813 TSSs had multiple PIRs. These PIRs collectively covered more than 90% of CGIs near or overlapping TSSs and included 15 types of repetitive elements (20% were LINE, 48% SINE, and the remaining 32% other types). In 75.3% of PIRs, the TSS was located inside the CGI. In the remaining PIRs, CGIs were very close to TSS (median distance is 295 bps). Conversely, repeats were well separated from CGIs, with an average distance of 825 bps.

Given a PIR trio, it was hypothesized that a functional methylation barrier in the tagged area could protect the promoter and the CGI against methylation spreading from the hypermethylated repeat; and a compromised barrier could lead to elevated methylation of the promoter and the CGI. To identify PIRs showing such methylation patterns, whole-genome bisulfite sequencing (WGBS) data from a previous study of colorectal cancers was analyzed. This data set included 3 pairs of matched normal and cancer samples and 23 unmatched cancer samples. For each PIR trio, a mixed-effect linear regression model was built to examine how the methylation level of a genomic position varied by its location (promoter, CGI, and repeat) and by the sample type (normal vs. cancer). Specifically, it was tested if the promoter and the CGI as compared to the repeat were hypomethylated in normal samples (i.e., under protection of a methylation barrier) and if their methylation levels increased significantly in cancers (i.e., loss of protection, FIG. 1B). At a false discovery rate <0.1 and a fold change >4, such patterns were observed in 2,252 trios and these were termed differentially methylated PIRs (dmPIRs). Overall, promoters and CGIs in dmPIRs were unmethylated in normal samples but became methylated in cancer samples (mean beta-value=2.8% vs. 19.0% for promoters, and 2.8% vs. 18.9% for CGIs), while repeats were consistently and heavily methylated in both normal and cancer samples (mean beta-value=78.5% and 71.0%, respectively, FIG. 1C).

A dmPIR tagged a genomic region bounded by a TSS and a repeat. To examine change of methylation level from repeats to TSS, each dmPIR-tagged region was divided into 20 consecutive windows and plotted the mean methylation level of CpG sites in each window. It was found that the decrease of methylation level from repeats to TSSs was a gradual transition instead of an abrupt drop (FIG. 1D). This pattern is consistent with previous reports of progressive methylation spreading. Furthermore, the figure showed that methylation spreading to TSS was more severe in cancer samples than in normal samples, as expected for compromised methylation barriers.

### A DNA Motif was Enriched in Protected Regions and was Homologous to the HS4 Methylation Barrier.

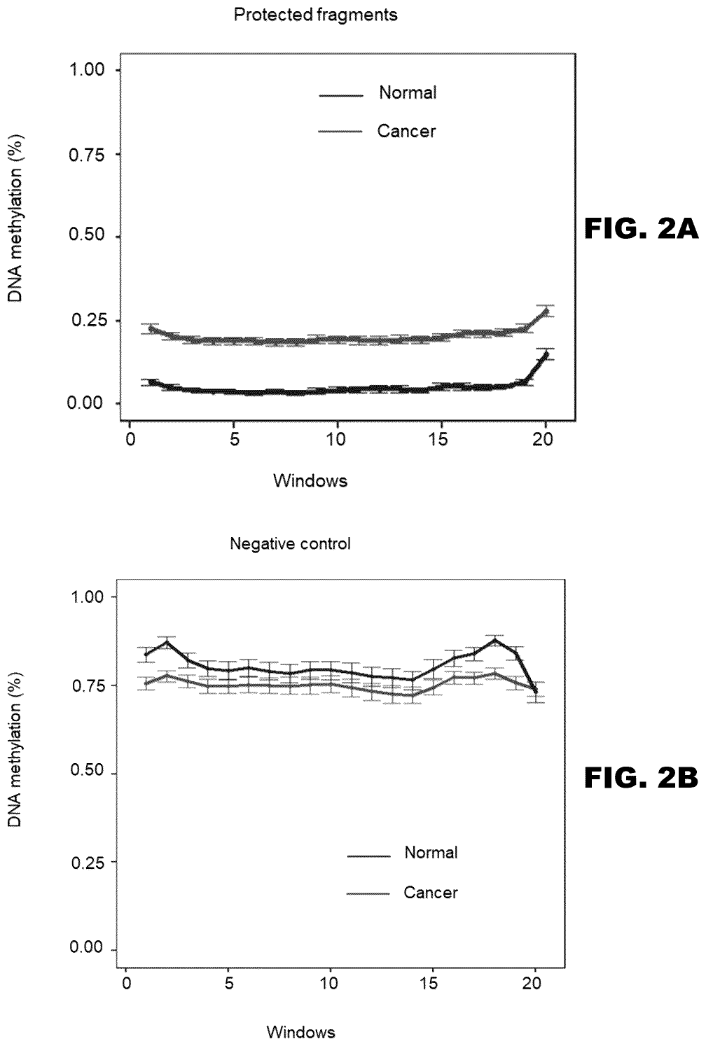

Each TSS tagged by a dmPIR was examined at the ±2 kbps flanking region to find the boundary of the segment protected from methylation in normal samples. It was hypothesized that these protected segments harbored methylation barriers and these barriers share common sequence signatures. Specifically, the ±2 kbps TSS-flanking region was divided into 40 sliding windows (window size=200 bps, step size=100 bps). Windows with a mean methylation level <0.1 in normal samples were considered unmethylated. The most upstream and the most downstream unmethylated windows marked the methylation boundary. Next, a set of non-dmPIRs was compiled as negative controls. Using the mixed-effect regression model, 1,342 trios were identified where promoters, CGIs, and repeats were consistently and heavily methylated in normal and cancer samples (mean Beta-value >70% in all groups, no significant difference between location or between sample type at nominal p >0.1). These non-dmPIRs-tagged regions contained no methylation barriers. To avoid over-representation of genomic regions with multiple TSSs, CGIs, or repeats, overlapping PIRs were merged, which produced 542 protected segments from dmPIRs and 532 unprotected segments from non-dmPIRs (FIGS. 2A-2B).

Using the MEME program, sequence motifs that were enriched in protected segments compared to unprotected segments were searched. A single motif with a significant E-value of 10-244 was found. This motif is 41-bps long and is C-rich (denoted as MB-41, FIG. 1E). It occurred in protected segments 4.0 times as often as in unprotected segments (116 vs. 29 occurrences per kbps). The q-value representing false discovery rate for an occurrence of this motif was 23.5 times lower in protected segments than in unprotected segments (median q-value=8.1×10⁻⁶ vs. 1.9×10⁻⁴, FIG. 1F).

The MB-41 motif was compared with the chicken HS4 element that is also C-rich. HS4 core element is a 239-bps long sequence consisting of five functional sites (FI to FV, FIG. 1G). Previous studies have shown that the 18-bp FIII site is the most important segment for preventing DNA methylation as deleting it caused complete loss of methylation protection function of HS4 while deleting other segments (FI and FV) caused only partial loss of function. The MB-41 motif was aligned to the HS4 sequence using the MAST program. It was found that MB-41 matched the full length of the FIII site and its flanking region (p-value <10−7, FIG. 1H). Therefore, MB-41 and the FIII methylation protector site in HS4 are homologous.

The dmPIRs and MB-41 Motif were Replicable in Independent Data Sets.

The WGBS data was used to discover dmPIRs because of the better coverage and higher resolution than data from methylation microarray or reduced representation bisulfite sequencing (RRBS). However, the small sample size of the WGBS data set and potential technical biases in library preparation and sequencing may affect the robustness of the results. Therefore, the reproducibility of the above findings were assessed in an independent RRBS data set.

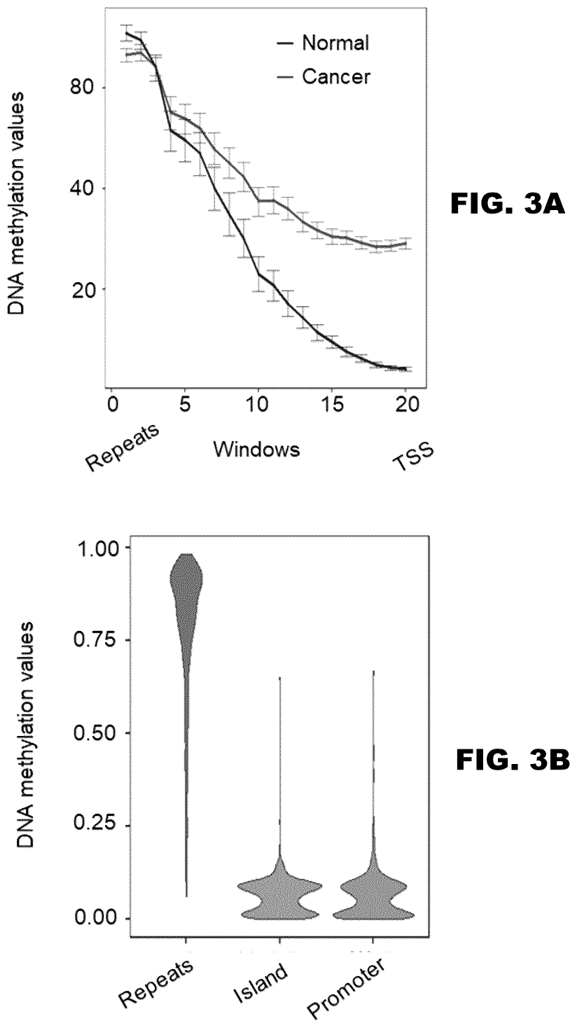

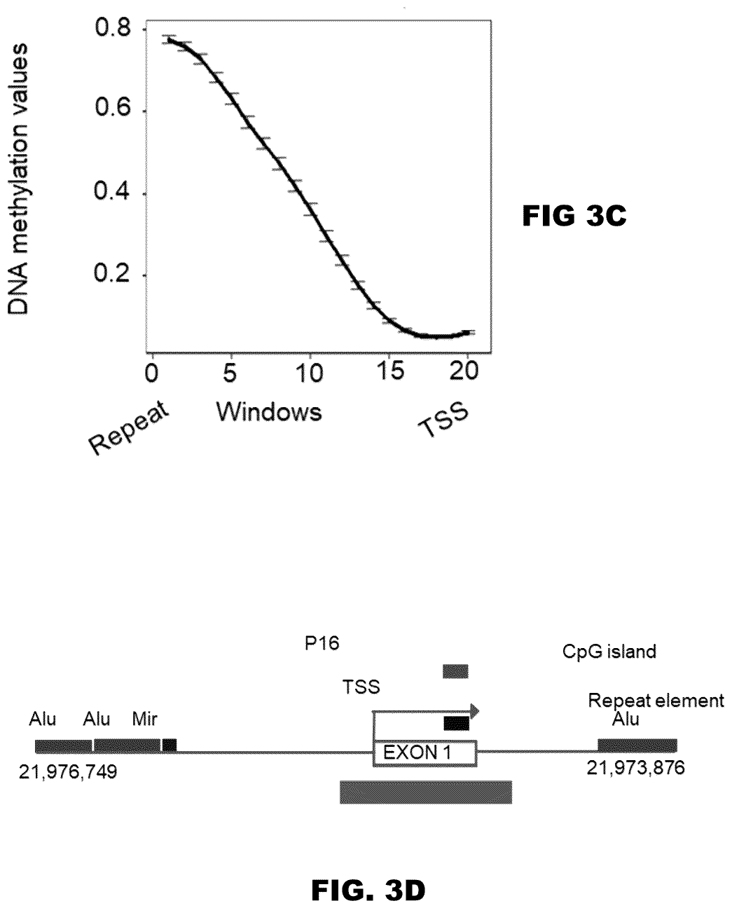

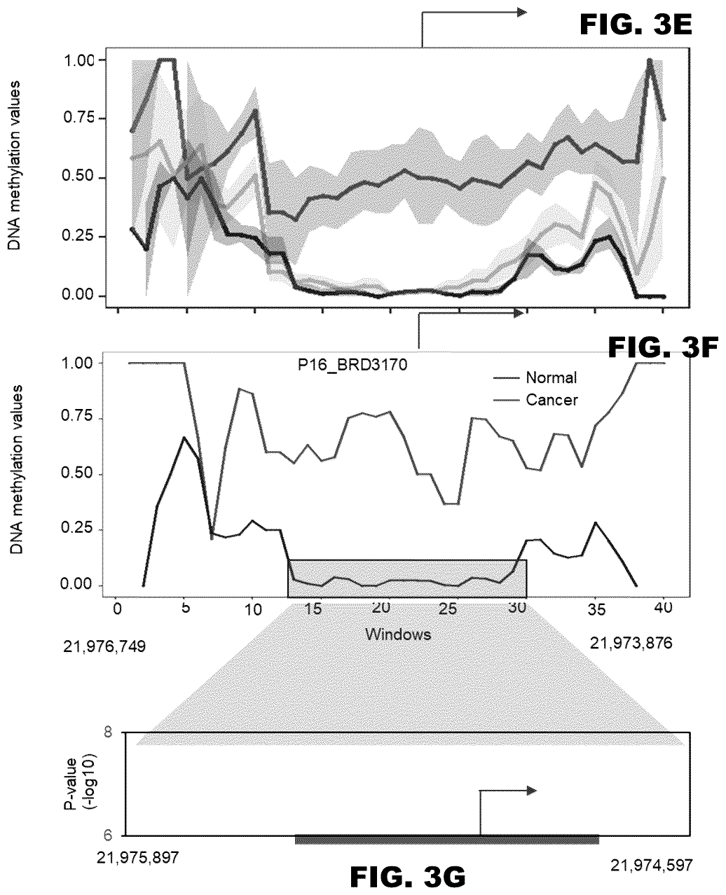

The RRBS data set was from a study of colorectal cancers that examined 9 pairs of matched normal-cancer samples. Due to the sparse coverage of RRBS, only less than half of the aforementioned dmPIRs (1,004 out of 2,252) had adequate methylation data (reported in at least three samples) for statistical analysis. We performed the same mixed-effect regression analysis using the RRBS data. For 64.7% (620) of the testable dmPIRs, the results from analyzing the RRBS data set were consistent with those from analyzing the WGBS data (FIG. 3A). In protected segmented tagged by these reproducible dmPIRs, the MB-41 motif had 165.5 occurrences per kbps, which was 5.7 times as often as in the unprotected segments (29 occurrences per kbps). The median q-value was 8.6×10−5. These samples also had RNA-seq data, allowing to examine if genes with aberrantly hypermethylated promoters were down-regulated. After mapping the 620 reproducible dmPIRs to 500 unique transcripts, it was observed a significant negative correlation between the fold change of promoter methylation level with the fold change of gene transcription level (Spearman's rank correlation rho=−0.16, p=3.4×10⁻⁴), confirming epigenetic regulation.

WGBS data of 11 normal gastric tissue samples from the ENCODE Project were further examined. It was confirmed that promoters and CGIs in 96.4% (2,171) of the dmPIRs had significantly lower methylation level than repeats (mean Beta-value=6.4%, 6.2%, and 77.9%, respectively, all FDR<0.1, FIG. 3B-C), supporting consistent protection from methylation in normal samples.

Selecting dmPIRs Containing the P16 Promoter for Functional Validation

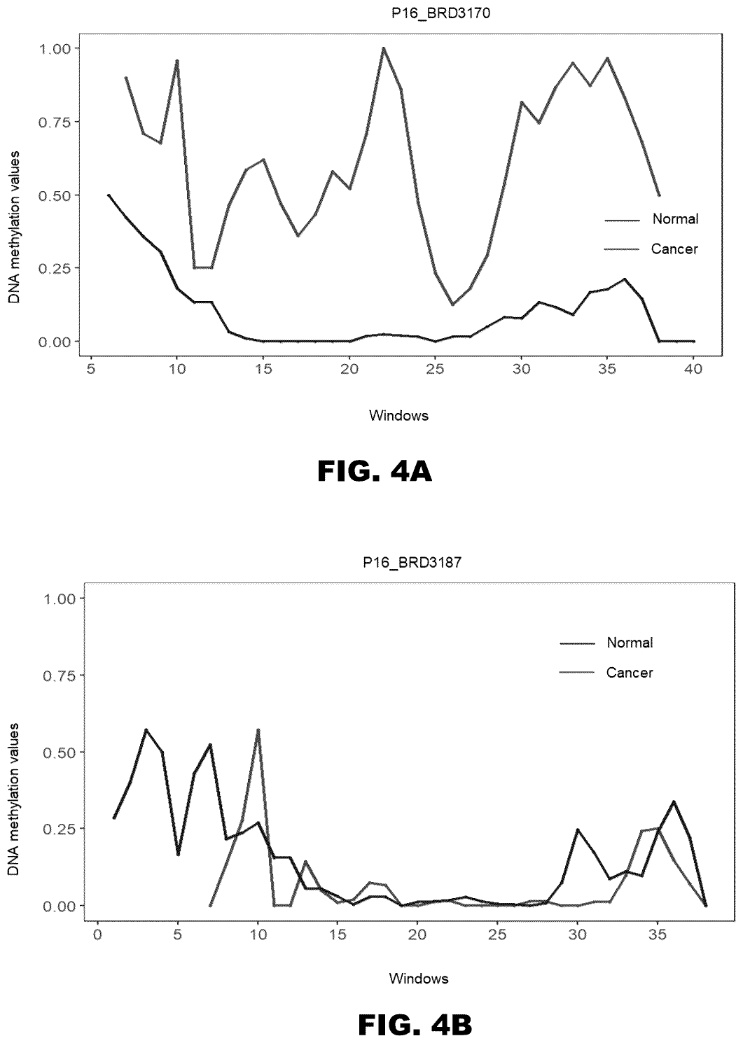

P16 is an important tumor suppressor gene, for which silencing via hypermethylation is a well-known mechanism of carcinogenesis. The TSS of the P16 gene is surrounded by four repetitive elements within the ±2 kbps flanking region. Three of them are Alu repeats and the other one is a mammalian-wide interspersed repeat (MIR, FIG. 3D). Of great interest, two dmPIRs each corresponding to a different Alu repeat were reproducibly found in the independent WGBS and RRBS data sets. The PIR trio corresponding to the third Alu repeat also showed a methylation spreading pattern although the FDR was borderline (0.13). The MIR contained only a single CpG site, thus was insufficient for statistical analysis. As expected, methylation level of the region spanning these four PIR trios followed a deep U-shaped curve in normal samples, showing a clear boundary separating the almost completely unmethylated center segment corresponding to the promoter and CGI from the left and right segments corresponding to the heavily methylated repeats (FIG. 3E-3F, and FIG. 4A-4B). This boundary was lost in 6 cancer samples, in which the methylation level of the promoter and CGI region elevated from nearly 0% in normal samples to higher than 20% in cancer samples. Interestingly, the methylation boundary remained largely intact in 20 cancer samples, indicating inter-tumor heterogeneity.

This region was scanned for the MB-41 motif using the MAST program and found 6 matching sites. All of these sites were inside the center segment protected from methylation in normal samples. The most significant site was located 64-105 bps upstream of the TSS (p-value=4.0×10−7, FIG. 3G). Four sites were clustered towards the right end of the protected area. These results suggested that the P16 gene was a good model for functional validation of methylation barrier and MB-41 matching sites.

### Targeted Sequencing Fine-Mapped Methylation Protection Boundaries in P16.

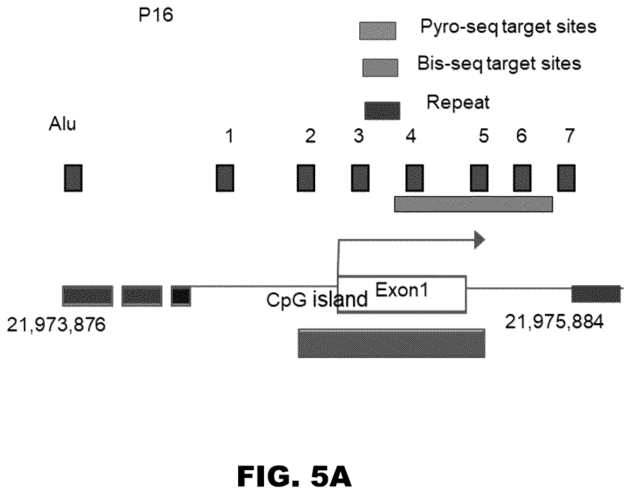

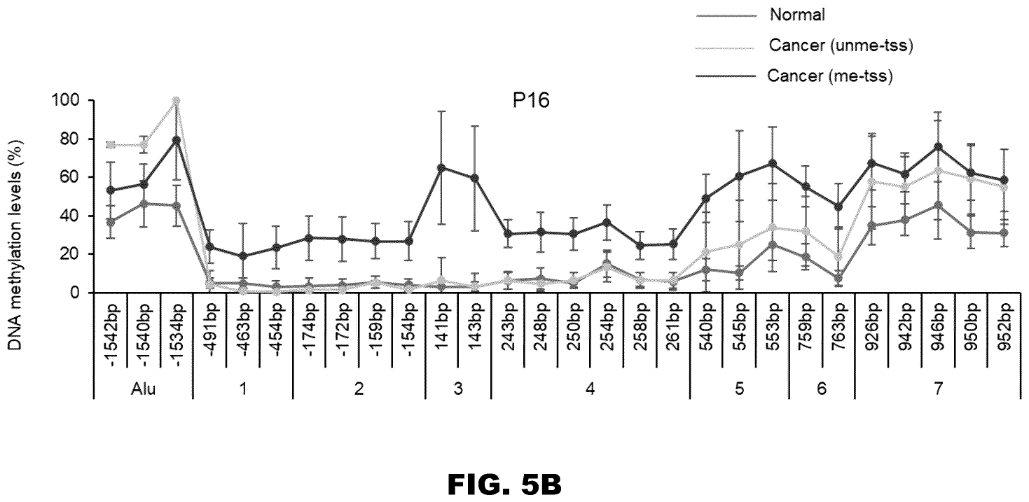

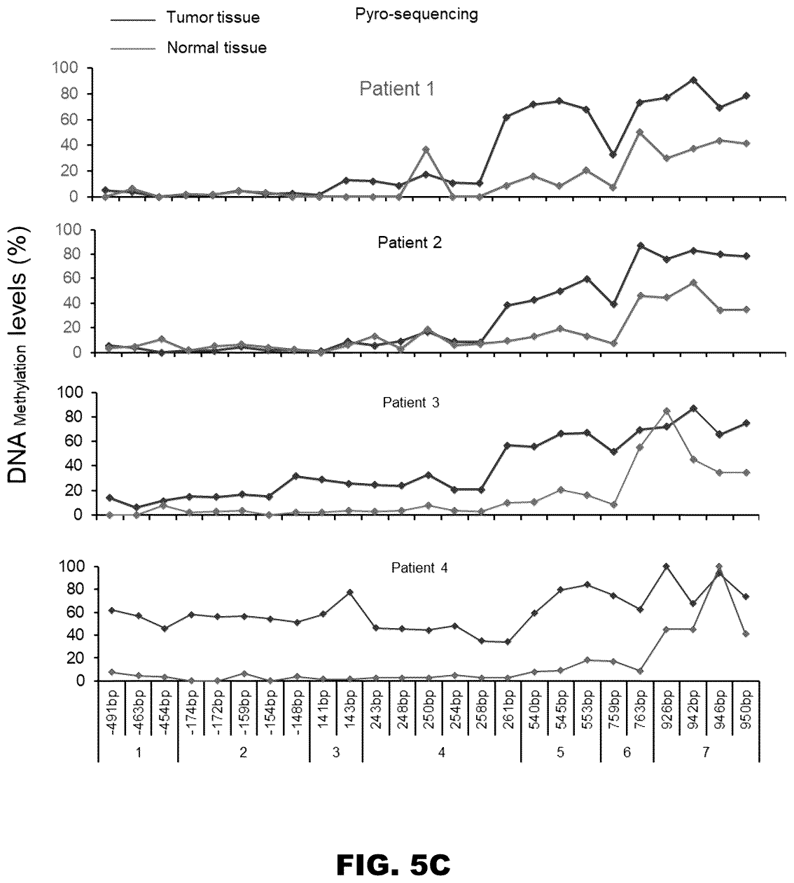

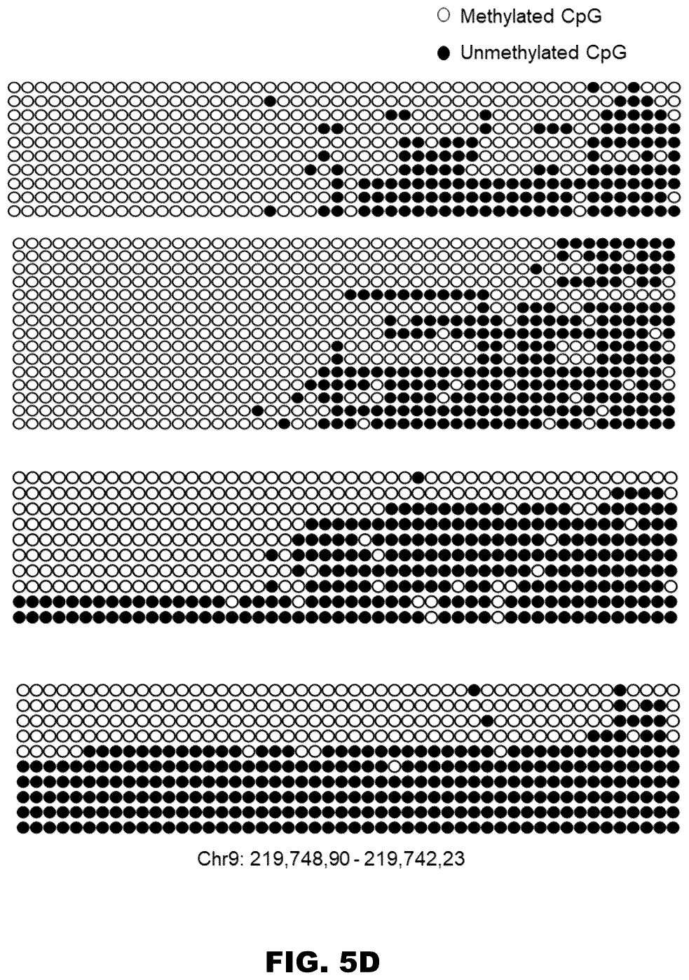

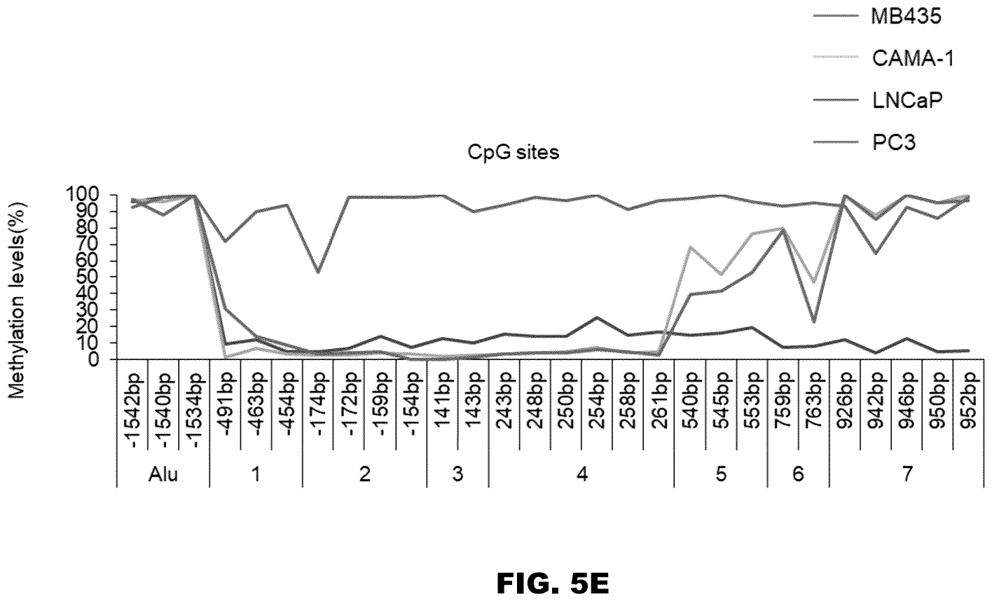

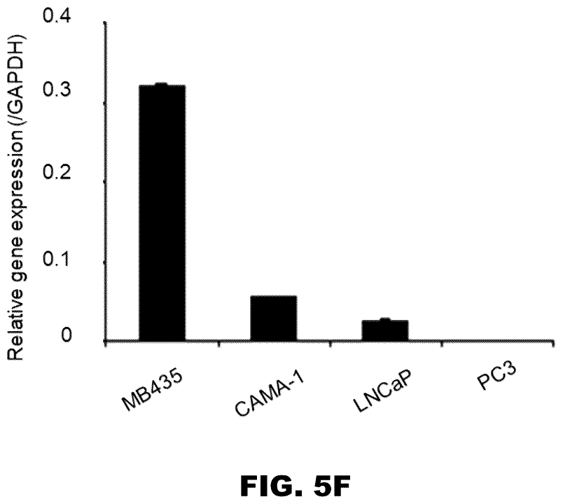

To confirm the methylation patterns in the P16 gene, targeted bisulfite-pyrosequencing was performed on 15 normal colon samples and 13 colon cancer samples. Eight target sites were selected, among which the Alu site and part of site #7 were mapped to repeats, sites #2 to #5 were mapped to the CGI, and sites #1 and #6 were mapped to regions between repeats and CGI (FIG. 5A). Consistent with the WGBS and RRBS data, target sites mapped to the repeats were heavily methylated in all samples (FIG. 5B). The remaining target sites were unmethylated in all normal samples and a subset of cancer samples. In 3 caner samples, the methylation level of these sites was elevated to 20-60%, implying loss of protection.

To design in vitro assays for functional analysis, the approximate boundary of the protected region was needed. The quick drop of methylation level from the Alu site to site #1 indicated that the upstream boundary of the protected region was between the Alu end position (−752 bp) and site #1 start position (−491 bp). However, the gradual change of methylation level across sites #4 to #7 made it difficult to determine the downstream boundary of the protected region. Therefore, bisulfite-PCR cloning was used and sequenced to examine the methylation levels of 50 CpG sites spanning a region between the site #4 start position (+243 bp) and the site #7 end position (+952 bp). Using the cancer samples, it was found that the downstream boundary of the protected region was located at approximately +400 bp downstream of the TSS (FIG. 5C-5D).

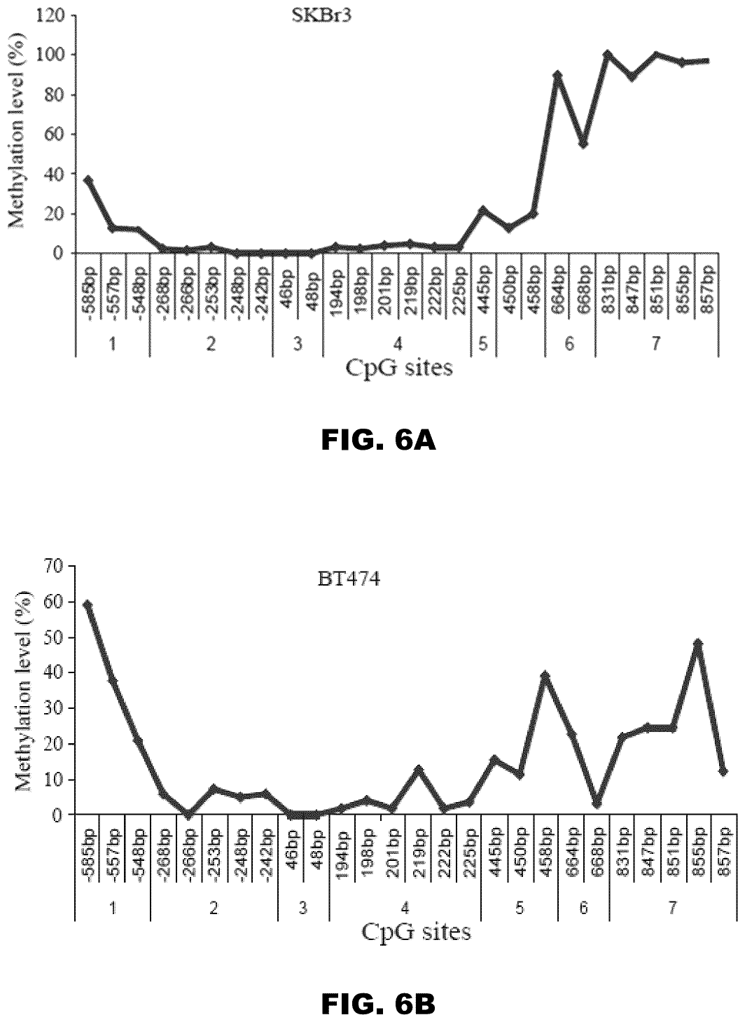

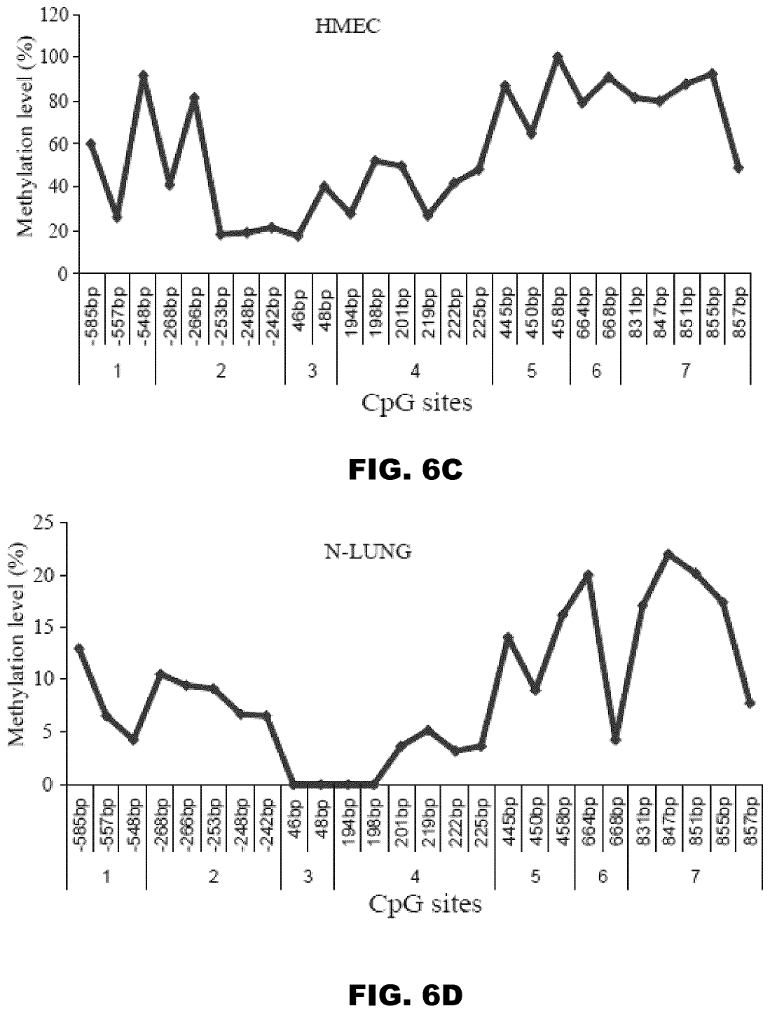

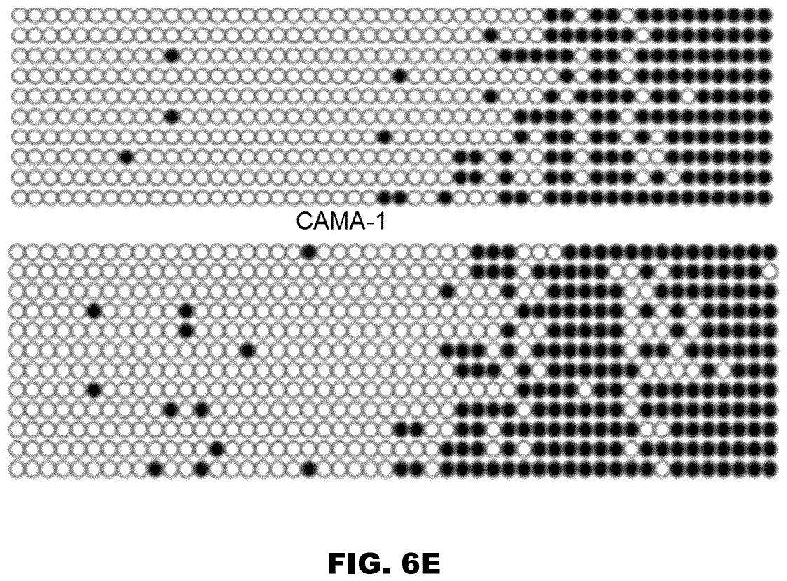

To find appropriate cell lines for in vitro assays, 19 cell lines from 7 different tissue types were screened for methylation levels in the P16 TSS-flanking region (Table 3). Among these cell lines, LNCaP and CAMA-1 showed methylation patterns like those observed in colon cancer patient samples (FIG. 5E & FIG. 6). We further examined the relationship between P16 promoter methylation and gene expression in these cell lines. In MB435 cells, the P16 TSS-flanking region was largely unmethylated and P16 was highly expressed. In PC3 cells, the P16 TSS-flanking region was uniformly hypermethylated and the P16 gene expression was undetectable. In LNCaP and CAMA-1 cells, the P16 TSS-flanking region was partially methylated and the P16 gene was expressed at intermediate levels. (FIG. 5E-F). This inverse relationship between methylation and expression confirmed that the P16 TSS-flanking region had epigenetic regulatory function. We thus used the LNCaP cell line for subsequent in vitro assays.

### In Vitro Experiments Functionally Validated the Methylation Protection Element

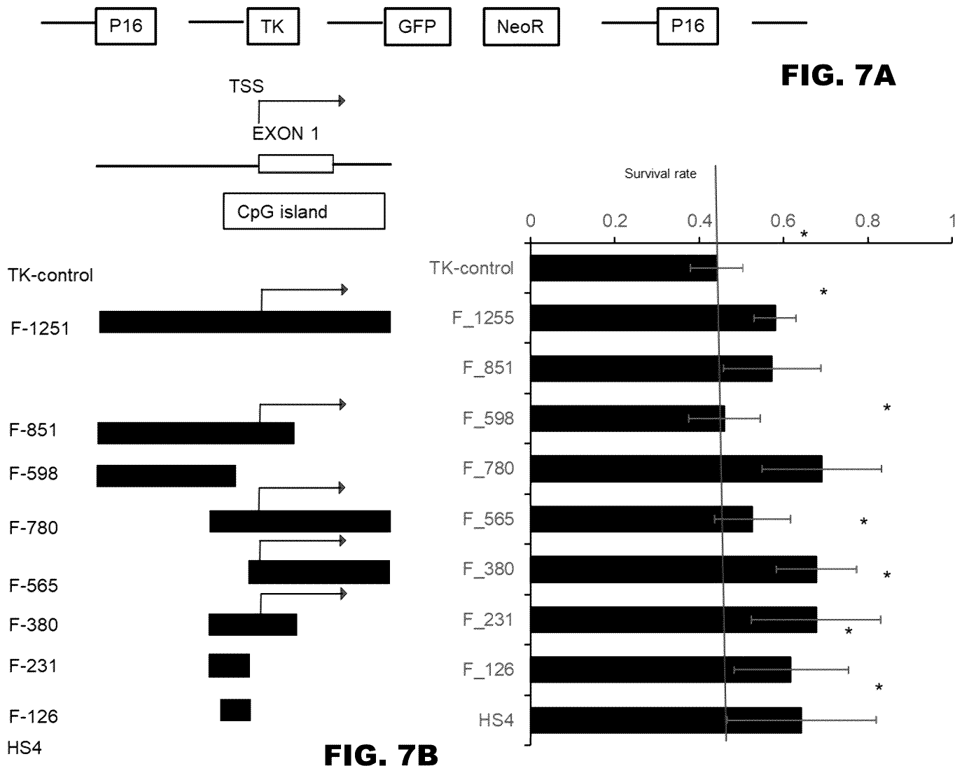

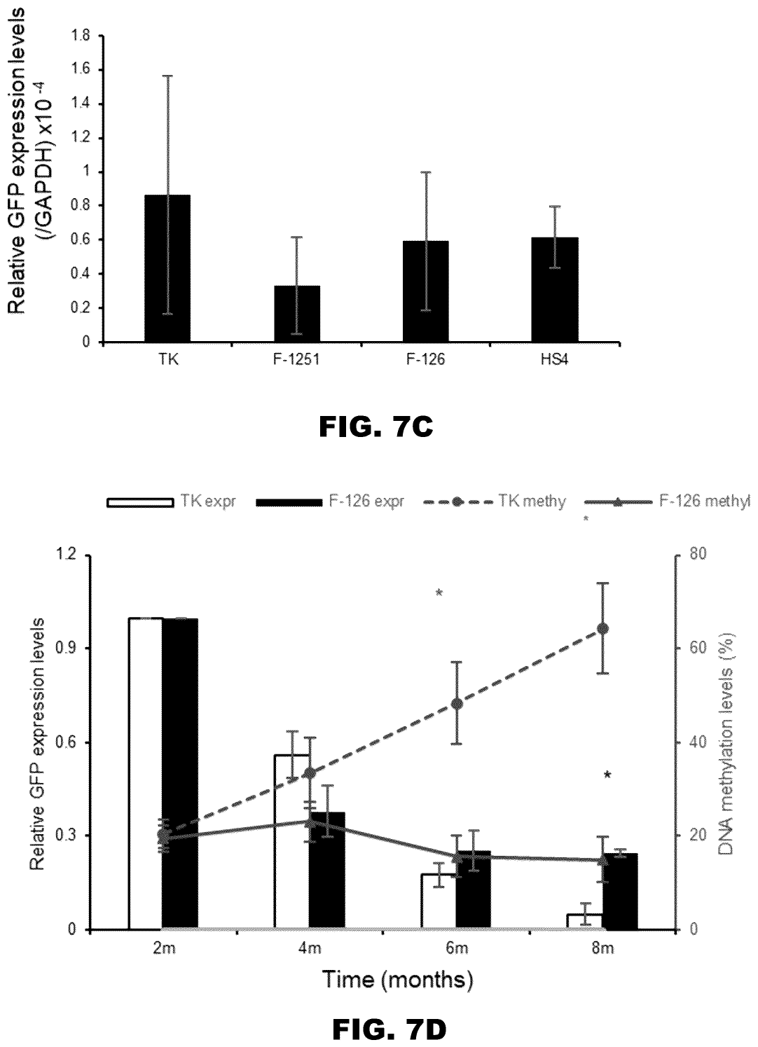

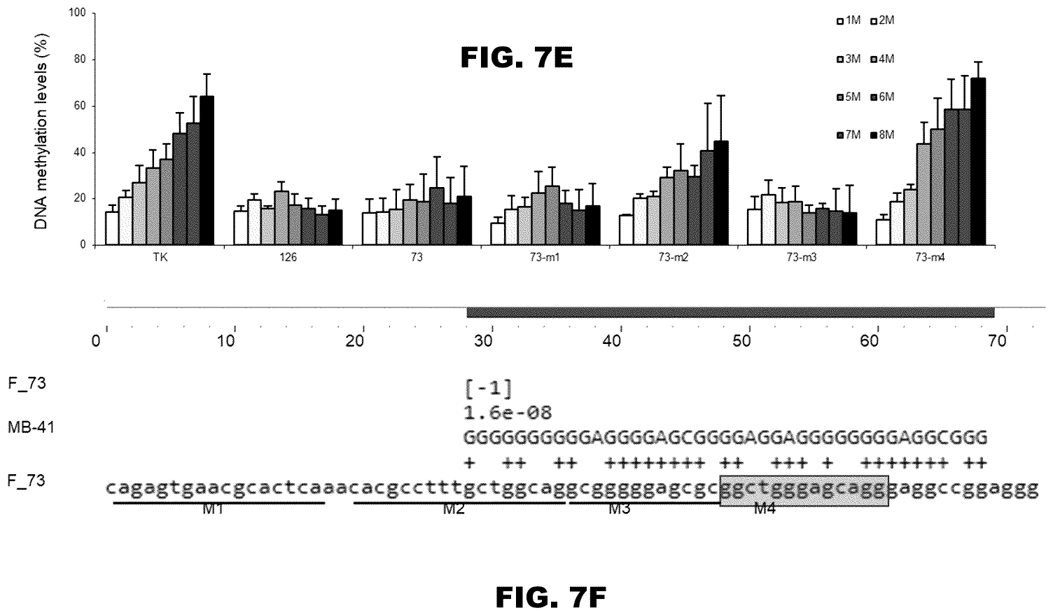

It was hypothesized that a cis-acting DNA segment protected the promoter of the P16 gene from methylation. Based on the upstream and downstream coordinates of the protected region, this segment was searched between −714 bp and +541 bp around the TSS (total 1,255 bps). Specifically, fragments were serially deleted from this region and designed a reporter system to test the effect (FIG. 7A). In this system, a fusion protein GFP-neo linking eGFP and the neomycin resistance gene was used as a reporter to measure the expression level with fluorescence intensity and surviving colony numbers after selection. The reporter was driven by a TK promoter, which is a CpG-rich promoter. Serial deletion fragments from the P16 protected region were placed at both sides of the reporter cassette.

The activity of the inserted fragments was measured by counting surviving colonies after extended neomycin selection. Cells transfected with these reporters were initially cultured with neomycin for one week and the number of surviving colonies was recorded as the baseline, reflecting stable integration. After the initial selection, cells were cultured without selection for two weeks. A second round of neomycin selection started 3 weeks after transfection to assess the protective effects of different fragments against epigenetic silencing, which was quantified by the ratio of neomycin resistant colonies after the second round of selection over that of the first round. Survival rate of constructs with different P16 deletion fragments was compared to that of the negative control (reporter without inserted P16 fragments). Each round of experiment was replicated four times.

Nine fragments were designed that covered various sections of the protected region (FIG. 7B, Table 2). The longest fragment F-1251 spanned the full length of the protected region. This fragment reported survival rate significantly higher than the negative control (mean=0.58 vs. 0.44, t test p=0.006), confirming the existence of a functional element. The fragment F-598 covered the first 598 bps of the protected region and reported survival rate similar to the control (mean=0.46 vs. 0.44, p=0.36), indicating the absence of a functional element. Among the fragments covering the remaining part of the protected region, F-231 was the shortest one reporting survival rate similar to that achieved by the full-length F-1251 fragment (mean=0.68). The F-126 fragment was part of F-231 and also reported survival rates significantly higher than the control (mean=0.62 p=0.014, FIG. 7B). Because the difference between F-126 and F-231 was not statistically significant (p=0.30), the functional element was plausibly located inside F-126.

The increased colony survival rates observed for the functional segments could be due to two reasons: a potential enhancer sequence and/or a transcriptional activator in these segments upregulated the expression of the fusion gene; alternatively, a potential methylation protection sequence in these segments prevented epigenetic silencing of the fusion gene.

The first possibility was ruled out by transiently transfecting the constructs into the LNCaP cells and measuring the expression level of GFPneo via real-time qPCR. Three fragments were tested that reported increased colony surviving ratios—the longest functional fragment F-1251, the shortest functional fragment F-126, and the chicken HS4 insulator. Reporter construct without inserted P16 fragments served as the negative control. No increase of GFPneo expression was observed for any of these inserts as compared to the negative control construct (FIG. 7C). Therefore, the increased survival rate was not due to enhancer and/or transcription activator effect.

To test the second possibility, long-term methylation changes and transgene expression at 2, 4, 6, and 8 months after transfection were analyzed. Three sites in TK promoter were selected for methylation measurement by bisulfite-pyrosequencing. It was found that in cells transfected with the negative control construct, DNA methylation steadily increased with the culture time (FIG. 7D). Conversely, methylation level remained largely unchanged over time in cells transfected with the F-126 functional construct. Meanwhile, real-time qPCR analysis showed that GFPneo expression decreased over time. However, such decrease was slower in cells infected with the F-126 construct than in cells infected with the negative control construct. After 8 months, gene expression level showed significant difference (FIG. 7D).

These results collectively supported that the F-126 fragment protected adjacent promoters from methylation and subsequently regulated gene transcription.

### Scanning Mutagenesis Fine-Mapped Functional Sites in the Protective Element.

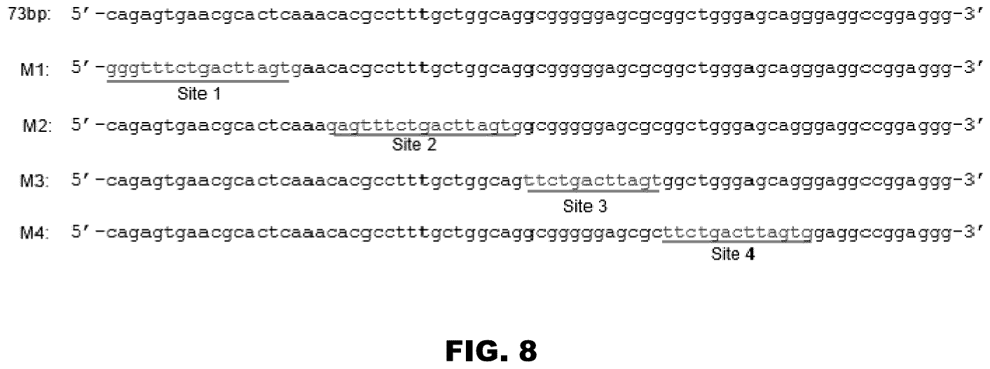

To identify functional sites in the protective element, a F-73 fragment was created that covered the first 73 bps of the F-126 fragment and contained a MB-41 matching site. As expected, cells transfected with F-73 constructs reported survival rate higher than the negative control (mean=0.60 vs. 0.47, p=0.001) and similar to that of F-126 constructs. The functional sites within the F-73 fragment were further fine-mapped using scanning mutagenesis. Specifically, 4 mutant oligonucleotides were synthesized (M1-M4, FIG. 8) each with 13-20 bps in the F-73 fragment replaced with a null sequence. This null sequence was selected from the F-598 fragment that showed no protective function (FIG. 5B) and had no significant protein binding sites according to JASPAR transcription factor binding site annotations in the UCSC Genome Browser. The null sequence had the same length of the replaced fragment.

Using the TK-GFPneo reporter system, methylation level of the TK promoter was monitored in cells transfected with the M1-M4 constructs. These cells were grown in neomycin-free media after the initial 1-week selection, and the DNA methylation status of the transfected cells was measured monthly for 8 months by bisulfite-pyrosequencing. Again, reporter construct without inserted P16 fragments was used as negative control, reporters flanked by the F-126 fragment, or the F-73 fragment were used as positive controls. After an eight-month culture, DNA methylation level of the TK promoter in the negative control group steadily increased from ˜20% to ˜80% (FIG. 7E). As expected, for constructs containing the F-126 or F-73 fragment, the DNA methylation level of the TK promoter remained below 20% throughout the 8-month culture time, confirming the protective function. The M1 and M3 mutants showed patterns similar to the unmutated F-73 fragment, suggesting that the substitutions in these two mutants had no functional impact (FIG. 7E). Conversely, the M4 mutant showed patterns similar to the negative TK control, suggesting the substitution in this mutant completely abolished the protective function. The M2 mutant also showed patterns indicating reduced protection, although the magnitude was not as strong as the M4 mutant. These results implied that the 13-bp sequence mutated in M4 and 17-bp sequence mutated in M2 were functional sites in the protective element, with the M4 segment being the most critical.

### The MB-41 Motif Matched the Functional Sites in the Protective Element

The F-73 fragment carried the MB-41 motif (p-value=1.6×10⁻⁸). The matched region included the entire functionally critical M4 site and part of the functional M2 site (FIG. 7F). The non-functional M1 site was outside the matched region. Therefore, the MB-41 motif correctly captured sequence features of the functional sites. These results, along with homology to the HS4 FIII site (FIG. 1G), collectively supported that the MB-41 motif was a sequence signature of DNA methylation protective elements.

Using the TOMTOM program, it was predicted that the MB-41 motif contained 35 transcription factor binding sites (TFBS, q-value <0.1, Table 4). These TFBS included binding sites of SP1, SP2, SP3, USF2, and VEZF1 that have been shown to bind to the chicken HS4 Fill site and putative methylation barriers in other species.

TABLE 4
35 candidate binding proteins on MB-41 motif by TOMTOM analysis (q-value <0.1).

| | | Opt- | | | | | | Target | |
|---|---|---|---|---|---|---|---|---|---|
| | | imal | | | | Over- | Query | con- | Orient- |
| Query ID | Target ID | offset | p−value | E−value | q−value | lap | consensus | sensus | ation |
| CCCSSCYCCY | SP2_HUMAN. | −19 | 1.25E−11 | 4.99E−09 | 9.57E−09 | 22 | CCCGCCCCC | CCCCCGGC | − |
| SCSCCBSCBC | H11MO.0.A | | | | | | CCCCCCCCC | CCCGCCCC | |
| CSCSCBCYCC | | | | | | | CCCCCGCCC | CCCCCC | |
| KCCCCSSSCS | | | | | | | CCCGCCCCC | | |
| C | | | | | | | CCCCC | | |
| CCCSSCYCCY | SP3_HUMAN. | −20 | 8.91E−11 | 3.57E−08 | 2.52E−08 | 20 | CCCGCCCCC | CCCCGGCC | − |
| SCSCCBSCBC | H11MO.0.B | | | | | | CCCCCCCCC | CCGCCCCC | |
| CSCSCBCYCC | | | | | | | CCCCCGCCC | CCCC | |
| KCCCCSSSCS | | | | | | | CCCGCCCCC | | |
| C | | | | | | | CCCCC | | |
| CCCSSCYCCY | PATZ1_HUMAN. | −19 | 1.16E−10 | 4.66E−08 | 2.52E−08 | 22 | CCCGCCCCC | CCTCCCCC | − |
| SCSCCBSCBC | H11MO.0.C | | | | | | CCCCCCCCC | CCCGCCCC | |
| CSCSCBCYCC | | | | | | | CCCCCGCCC | CTCCCC | |
| KCCCCSSSCS | | | | | | | CCCGCCCCC | | |
| C | | | | | | | CCCCC | | |
| CCCSSCYCCY | MAZ_HUMAN. | −14 | 1.31E−10 | 5.27E−08 | 2.52E−08 | 22 | CCCGCCCCC | CCCCCCCC | − |
| SCSCCBSCBC | H11MO.0.A | | | | | | CCCCCCCCC | CCCCCCCC | |
| CSCSCBCYCC | | | | | | | CCCCCGCCC | TCCCCC | |
| KCCCCSSSCS | | | | | | | CCCGCCCCC | | |
| C | | | | | | | CCCCC | | |
| CCCSSCYCCY | ZN467_HUMAN. | −13 | 2.40E−10 | 9.61E−08 | 3.68E−08 | 22 | CCCGCCCCC | CCCCCCCC | − |
| SCSCCBSCBC | H11MO.0.C | | | | | | CCCCCCCCC | CCCCTCCC | |
| CSCSCBCYCC | | | | | | | CCCCCGCCC | CTCCCC | |
| KCCCCSSSCS | | | | | | | CCCGCCCCC | | |
| C | | | | | | | CCCCC | | |
| CCCSSCYCCY | WT1_HUMAN. | −20 | 1.29E−09 | 5.18E−07 | 1.43E−07 | 20 | CCCGCCCCC | CCCCCCCT | − |
| SCSCCBSCBC | H11MO.0.C | | | | | | CCCCCCCCC | CCTCCCCC | |
| CSCSCBCYCC | | | | | | | CCCCCGCCC | GCCC | |
| KCCCCSSSCS | | | | | | | CCCGCCCCC | | |
| C | | | | | | | CCCCC | | |
| CCCSSCYCCY | SP1_HUMAN. | −15 | 1.30E−09 | 5.22E−07 | 1.43E−07 | 22 | CCCGCCCCC | CCCCCCCC | − |
| SCSCCBSCBC | H11MO.0.A | | | | | | CCCCCCCCC | CGGCCCCG | |
| CSCSCBCYCC | | | | | | | CCCCCGCCC | CCCCCC | |
| KCCCCSSSCS | | | | | | | CCCGCCCCC | | |
| C | | | | | | | CCCCC | | |
| CCCSSCYCCY | KLF15_HUMAN. | −16 | 3.47E−09 | 1.39E−06 | 3.34E−07 | 19 | CCCGCCCCC | CCCCCCCC | − |
| SCSCCBSCBC | H11MO.0.A | | | | | | CCCCCCCCC | TGCTCCTC | |
| CSCSCBCYCC | | | | | | | CCCCCGCCC | CCC | |
| KCCCCSSSCS | | | | | | | CCCGCCCCC | | |
| C | | | | | | | CCCCC | | |
| CCCSSCYCCY | VEZF1_HUMAN. | −5 | 1.35E−08 | 5.41E−06 | 1.15E−06 | 22 | CCCGCCCCC | CCCCTCCC | − |
| SCSCCBSCBC | H11MO.0.C | | | | | | CCCCCCCCC | CCTCCCCC | |
| CSCSCBCYCC | | | | | | | CCCCCGCCC | CTCCCC | |
| KCCCCSSSCS | | | | | | | CCCGCCCCC | | |
| C | | | | | | | CCCCC | | |
| CCCSSCYCCY | ZN263_HUMAN. | −14 | 7.94E−08 | 3.18E−05 | 6.10E−06 | 20 | CCCGCCCCC | CTCCTCCT | − |
| SCSCCBSCBC | H11MO.0.A | | | | | | CCCCCCCCC | CTCCCTCC | |
| CSCSCBCYCC | | | | | | | CCCCCGCCC | TCCC | |
| KCCCCSSSCS | | | | | | | CCCGCCCCC | | |
| C | | | | | | | CCCCC | | |
| CCCSSCYCCY | ZN341_HUMAN. | −1 | 4.95E−07 | 0.000198496 | 3.46E−05 | 22 | CCCGCCCCC | GCTCTTCC | − |
| SCSCCBSCBC | H11MO.0.C | | | | | | CCCCCCCCC | CTCCCCCC | |
| CSCSCBCYCC | | | | | | | CCCCCGCCC | CCCCCC | |
| KCCCCSSSCS | | | | | | | CCCGCCCCC | | |
| C | | | | | | | CCCCC | | |
| CCCSSCYCCY | KLF3_HUMAN. | 0 | 2.21E−06 | 0.000884859 | 0.000141351 | 19 | CCCGCCCCC | CCCGGCCC | − |
| SCSCCBSCBC | H11MO.0.B | | | | | | CCCCCCCCC | CGCCCCTC | |
| CSCSCBCYCC | | | | | | | CCCCCGCCC | CCC | |
| KCCCCSSSCS | | | | | | | CCCGCCCCC | | |
| C | | | | | | | CCCCC | | |
| CCCSSCYCCY | SP4_HUMAN. | 0 | 5.45E−06 | 0.00218442 | 0.000322105 | 20 | CCCGCCCCC | CCCGGCCC | − |
| SCSCCBSCBC | H11MO.0.A | | | | | | CCCCCCCCC | CGCCCCCT | |
| CSCSCBCYCC | | | | | | | CCCCCGCCC | TCCC | |
| KCCCCSSSCS | | | | | | | CCCGCCCCC | | |
| C | | | | | | | CCCCC | | |
| CCCSSCYCCY | KLF6_HUMAN. | −19 | 1.89E−05 | 0.0075942 | 0.00103982 | 19 | CCCGCCCCC | CCCCCGGC | − |
| SCSCCBSCBC | H11MO.0.A | | | | | | CCCCCCCCC | CCCGCCCT | |
| CSCSCBCYCC | | | | | | | CCCCCGCCC | TCC | |
| KCCCCSSSCS | | | | | | | CCCGCCCCC | | |
| C | | | | | | | CCCCC | | |
| CCCSSCYCCY | EGR1_HUMAN. | −25 | 2.63E−05 | 0.010563 | 0.0013499 | 16 | CCCGCCCCC | CCCCCGCC | − |
| SCSCCBSCBC | H11MO.0.A | | | | | | CCCCCCCCC | CACGCCCT | |
| CSCSCBCYCC | | | | | | | CCCCCGCCC | C | |
| KCCCCSSSCS | | | | | | | CCCGCCCCC | | |
| C | | | | | | | CCCCC | | |
| CCCSSCYCCY | EGR2_HUMAN. | −5 | 4.16E−05 | 0.0166775 | 0.00194125 | 18 | CCCGCCCCC | CCCCTCCC | − |
| SCSCCBSCBC | H11MO.0.A | | | | | | CCCCCCCCC | ACACCCCC | |
| CSCSCBCYCC | | | | | | | CCCCCGCCC | CC | |
| KCCCCSSSCS | | | | | | | CCCGCCCCC | | |
| C | | | | | | | CCCCC | | |
| CCCSSCYCCY | ZN281_HUMAN. | −25 | 4.29E−05 | 0.0172158 | 0.00194125 | 15 | CCCGCCCCC | TCCCCTCC | − |
| SCSCCBSCBC | H11MO.0.A | | | | | | CCCCCCCCC | CCCACCC | |
| CSCSCBCYCC | | | | | | | CCCCCGCCC | | |
| KCCCCSSSCS | | | | | | | CCCGCCCCC | | |
| C | | | | | | | CCCCC | | |
| CCCSSCYCCY | E2F1_HUMAN. | −16 | 8.21E−05 | 0.0329331 | 0.00350724 | 14 | CCCGCCCCC | CTTTCCCG | − |
| SCSCCBSCBC | H11MO.0.A | | | | | | CCCCCCCCC | CCCCCC | |
| CSCSCBCYCC | | | | | | | CCCCCGCCC | | |
| KCCCCSSSCS | | | | | | | CCCGCCCCC | | |
| C | | | | | | | CCCCC | | |
| CCCSSCYCCY | FLI1_HUMAN. | −18 | 0.000106869 | 0.0428545 | 0.00432362 | 18 | CCCGCCCCC | TCCCTCCT | − |
| SCSCCBSCBC | H11MO.0.A | | | | | | CCCCCCCCC | TCCTTCCT | |
| CSCSCBCYCC | | | | | | | CCCCCGCCC | CC | |
| KCCCCSSSCS | | | | | | | CCCGCCCCC | | |
| C | | | | | | | CCCCC | | |
| CCCSSCYCCY | ZBT17_HUMAN. | −8 | 0.000112722 | 0.0452015 | 0.00433239 | 19 | CCCGCCCCC | CTTCCCCT | − |
| SCSCCBSCBC | H11MO.0.A | | | | | | CCCCCCCCC | CCCCCACC | |
| CSCSCBCYCC | | | | | | | CCCCCGCCC | CTC | |
| KCCCCSSSCS | | | | | | | CCCGCCCCC | | |
| C | | | | | | | CCCCC | | |
| CCCSSCYCCY | E2F6_HUMAN. | −15 | 0.000135244 | 0.054233 | 0.0049505 | 13 | CCCGCCCCC | CCCTTCCC | − |
| SCSCCBSCBC | H11MO.0.A | | | | | | CCCCCCCCC | GCCCC | |
| CSCSCBCYCC | | | | | | | CCCCCGCCC | | |
| KCCCCSSSCS | | | | | | | CCCGCCCCC | | |
| C | | | | | | | CCCCC | | |
| CCCSSCYCCY | KLF1_HUMAN. | −27 | 0.000313112 | 0.125558 | 0.0109402 | 14 | CCCGCCCCC | CCCGGCCC | − |
| SCSCCBSCBC | H11MO.0.A | | | | | | CCCCCCCCC | CGCCCC | |
| CSCSCBCYCC | | | | | | | CCCCCGCCC | | |
| KCCCCSSSCS | | | | | | | CCCGCCCCC | | |
| C | | | | | | | CCCCC | | |
| CCCSSCYCCY | RXRA_HUMAN. | −6 | 0.000461743 | 0.185159 | 0.0154158 | 20 | CCCGCCCCC | CTCTGACC | − |
| SCSCCBSCBC | H11MO.0.A | | | | | | CCCCCCCCC | TCTGCCTC | |
| CSCSCBCYCC | | | | | | | CCCCCGCCC | CCCC | |
| KCCCCSSSCS | | | | | | | CCCGCCCCC | | |
| C | | | | | | | CCCCC | | |
| CCCSSCYCCY | KLF12_HUMAN. | −25 | 0.000481313 | 0.193006 | 0.0154158 | 11 | CCCGCCCCC | GCCCCGCC | − |
| SCSCCBSCBC | H11MO.0.C | | | | | | CCCCCCCCC | CCT | |
| CSCSCBCYCC | | | | | | | CCCCCGCCC | | |
| KCCCCSSSCS | | | | | | | CCCGCCCCC | | |
| C | | | | | | | CCCCC | | |
| CCCSSCYCCY | COT1_HUMAN. | −19 | 0.000642736 | 0.257737 | 0.0197625 | 17 | CCCGCCCCC | CCCTGACC | − |
| SCSCCBSCBC | H11MO.0.C | | | | | | CCCCCCCCC | TCTGACCC | |
| CSCSCBCYCC | | | | | | | CCCCCGCCC | C | |
| KCCCCSSSCS | | | | | | | CCCGCCCCC | | |
| C | | | | | | | CCCCC | | |
| CCCSSCYCCY | KLF9_HUMAN. | −3 | 0.000827132 | 0.33168 | 0.0244541 | 15 | CCCGCCCCC | GGCCACGC | − |
| SCSCCBSCBC | H11MO.0.C | | | | | | CCCCCCCCC | CCCCTCC | |
| CSCSCBCYCC | | | | | | | CCCCCGCCC | | |
| KCCCCSSSCS | | | | | | | CCCGCCCCC | | |
| C | | | | | | | CCCCC | | |
| CCCSSCYCCY | ZN770_HUMAN. | −1 | 0.00123701 | 0.496041 | 0.0352175 | 22 | CCCGCCCCC | GATCCTCC | − |
| SCSCCBSCBC | H11MO.0.C | | | | | | CCCCCCCCC | CGCCTCAG | |
| CSCSCBCYCC | | | | | | | CCCCCGCCC | CCTCCC | |
| KCCCCSSSCS | | | | | | | CCCGCCCCC | | |
| C | | | | | | | CCCCC | | |
| CCCSSCYCCY | E2F4_HUMAN. | −16 | 0.00158173 | 0.634274 | 0.0434234 | 13 | CCCGCCCCC | ATTTCCCG | − |
| SCSCCBSCBC | H11MO.0.A | | | | | | CCCCCCCCC | CCCCC | |
| CSCSCBCYCC | | | | | | | CCCCCGCCC | | |
| KCCCCSSSCS | | | | | | | CCCGCCCCC | | |
| C | | | | | | | CCCCC | | |
| CCCSSCYCCY | USF2_HUMAN. | −16 | 0.00185525 | 0.743955 | 0.049176 | 19 | CCCGCCCCC | CGCCGCG | − |
| SCSCCBSCBC | H11MO.0.A | | | | | | CCCCCCCCC | GCCACGTG | |
| CSCSCBCYCC | | | | | | | CCCCCGCCC | ACCC | |
| KCCCCSSSCS | | | | | | | CCCGCCCCC | | |
| C | | | | | | | CCCCC | | |
| CCCSSCYCCY | MXI1_HUMAN. | −17 | 0.00214357 | 0.859573 | 0.0549246 | 15 | CCCGCCCCC | CCCGCCGC | − |
| SCSCCBSCBC | H11MO.0.A | | | | | | CCCCCCCCC | CACGTGC | |
| CSCSCBCYCC | | | | | | | CCCCCGCCC | | |
| KCCCCSSSCS | | | | | | | CCCGCCCCC | | |
| C | | | | | | | CCCCC | | |
| CCCSSCYCCY | ASCL1_HUMAN. | −8 | 0.00230604 | 0.924722 | 0.0571814 | 14 | CCCGCCCCC | CTGCACCT | + |
| SCSCCBSCBC | H11MO.0.A | | | | | | CCCCCCCCC | GCTCCC | |
| CSCSCBCYCC | | | | | | | CCCCCGCCC | | |
| KCCCCSSSCS | | | | | | | CCCGCCCCC | | |
| C | | | | | | | CCCCC | | |
| CCCSSCYCCY | TFDP1_HUMAN. | −16 | 0.00246947 | 0.990257 | 0.0593202 | 14 | CCCGCCCCC | TTTTCCCG | − |
| SCSCCBSCBC | H11MO.0.C | | | | | | CCCCCCCCC | CCCCCC | |
| CSCSCBCYCC | | | | | | | CCCCCGCCC | | |
| KCCCCSSSCS | | | | | | | CCCGCCCCC | | |
| C | | | | | | | CCCCC | | |
| CCCSSCYCCY | E2F7_HUMAN. | −28 | 0.0026387 | 1.05812 | 0.0614647 | 13 | CCCGCCCCC | CCTTTCCC | − |
| SCSCCBSCBC | H11MO.0.B | | | | | | CCCCCCCCC | GCCCC | |
| CSCSCBCYCC | | | | | | | CCCCCGCCC | | |
| KCCCCSSSCS | | | | | | | CCCGCCCCC | | |
| C | | | | | | | CCCCC | | |
| CCCSSCYCCY | PTF1A_HUMAN. | −4 | 0.00308945 | 1.23887 | 0.069074 | 18 | CCCGCCCCC | CCAGCTGC | + |
| SCSCCBSCBC | H11MO.0.B | | | | | | CCCCCCCCC | CCCCTTTC | |
| CSCSCBCYCC | | | | | | | CCCCCGCCC | CC | |
| KCCCCSSSCS | | | | | | | CCCGCCCCC | | |
| C | | | | | | | CCCCC | | |
| CCCSSCYCCY | THAP1_HUMAN. | −17 | 0.00314509 | 1.26118 | 0.069074 | 22 | CCCGCCCCC | CGCCGCCA | + |
| SCSCCBSCBC | H11MO.0.C | | | | | | CCCCCCCCC | TCTTGGCT | |
| CSCSCBCYCC | | | | | | | CCCCCGCCC | GCGGGC | |
| KCCCCSSSCS | | | | | | | CCCGCCCCC | | |
| C | | | | | | | CCCCC | | |

Aberrant Methylation of Cancer Genes was Associated with dmPIRs and the MB-41 Motif.

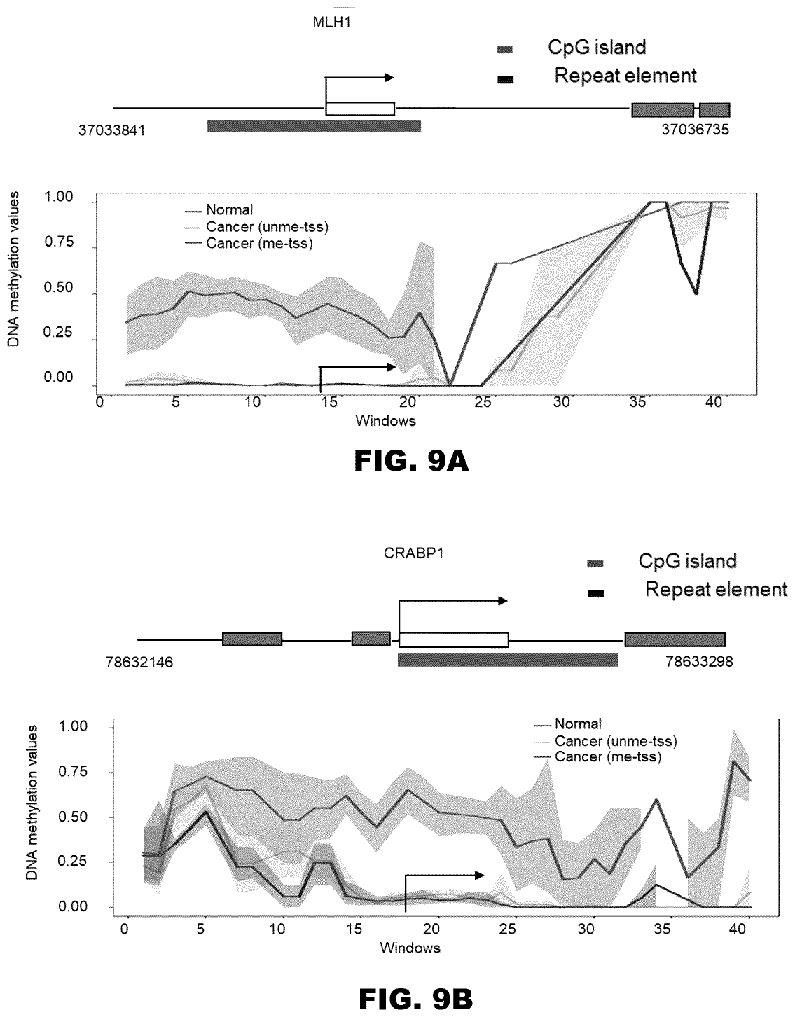

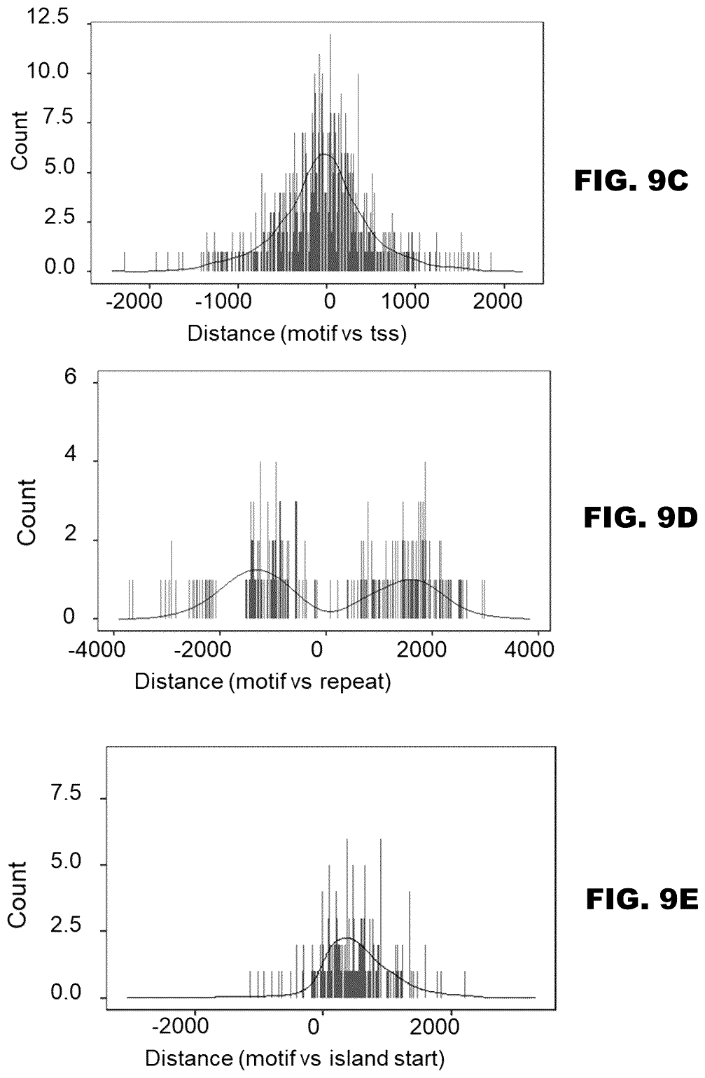

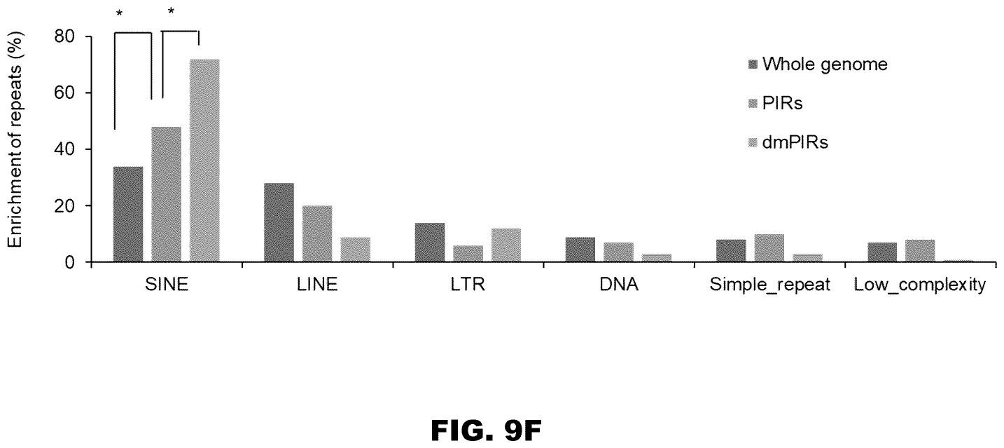

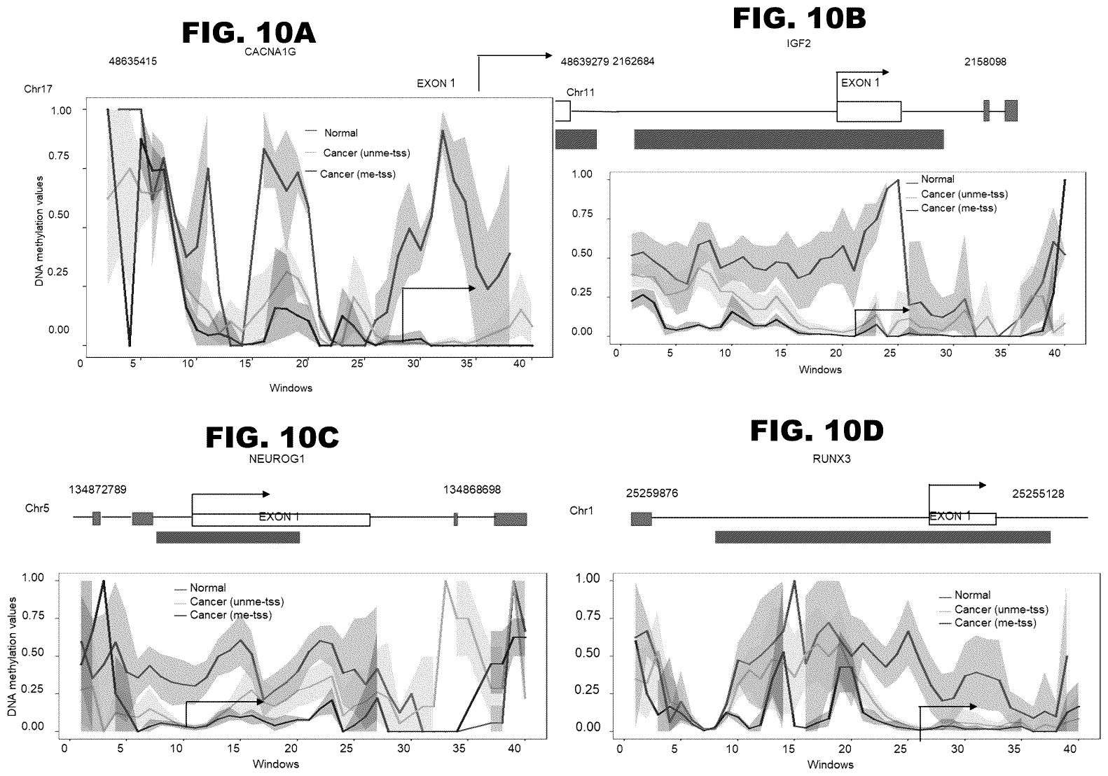

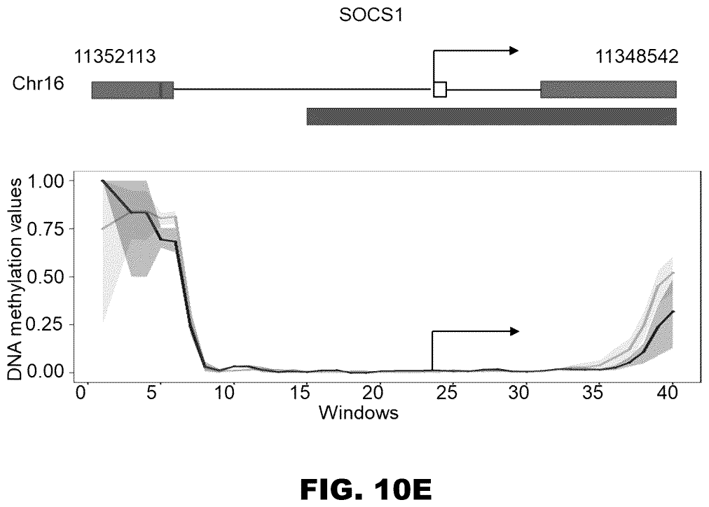

The dmPIRs contained TSSs of 610 unique genes, among which 93 genes were classified as tumor suppressors by the GUST program (probability >0.5). This was not surprising because the data sets we used to discover the dmPIRs were from colorectal cancers. In particular a subset of colorectal cancers displays CpG island methylator phenotype (CIMP) where hypermethylation of promoter-associated CGIs deactivates tumor suppressor genes, such as P16. Other commonly affected genes in CIMP include CRABP1, MLH1, CACNA1G, IGF2, NEUROG1, RUNX3 and SOCS1. Except SOCS1, all of these genes showed the characteristic pattern of methylation spreading from adjacent repeats to promoters in the WGBS samples (FIG. 9A-9B, and FIG. 10). Three genes, namely P16, CRABP1 and MLH1, were found in the dmPIRs. The rest of them were not included in the dmPIRs because the surrounding repeats were more than 2 kbps away from TSS. tumor suppressor genes

However, many genes unrelated to cancers were also unexpectedly tagged by dmPIRs. In fact, it was found no enrichment of cancer genes (FDR >0.1) but 2.4-fold enrichment of genes with RNA polymerase II-specific DNA-binding transcription factor activity (FDR=5.64 ×10⁻⁹) in dmPIRs. These results implied that the putative methylation barriers and the MB-41 motif identified likely influenced a wide range of cellular processes and did not specifically target cancer pathways.

MB-41 Motif was Located Closer to TSS than to Repeat and Away from Methylation Boundary.

Using the MAST program, the sequences matching the MB-41 motif in dmPIR-tagged regions were located. With p-value <0.0001, 2,704 occurrences were found. Overall, a MB-41 matching site was 273 bps (median) upstream or 309 bps downstream of a TSS (FIG. 9C), which was 4-5 times shorter than its distance to a repeat (1,440 bps, FIG. 9D). A majority (70.0%) of the MB-41 matching sites were located inside CGIs, which was expected as this motif is C-rich and the average length of CGIs was approximately 900 bps (FIG. 9E). These distributions implied that most methylation protective elements were close to TSS, within a CGI, and clearly separated from the flanking repetitive elements.

Then, the location of MB-41 motif was examined relative to methylation boundary in the 542 protected regions tagged by dmPIRs. Sequences matched to the MB-41 motif were 691 bps (median) away from the methylation boundary. Given that the protected regions have a median length of 1,300 bps (range 200-4100 bps), the methylation protective elements marked by the MB-41 motif were close to the middle of a protected fragment, instead of at the boundaries. The experimentally validated protective element in the P16 gene also conformed to the overall MB-41 distribution pattern. The functionally critical M4 site was 72 bps upstream to the TSS, protecting a genomic region of 1008 bps from methylation in normal samples. This element was ˜700 bps from the upstream MIR repeat and ˜1000 bps away from the downstream Alu repeat.

Repeats in dmPIRs were Enriched with SINEs.

The human genome contains 17 types of repetitive elements. The most abundant types are SINEs (34%) and LINEs (28%). Among the 381,180 PIRs identified in the human genome, all but one of the 17 types of repeats were represented, although not proportionally—SINEs were over-represented (48%, odds ratio=1.81, fisher test p=0) and LINEs were under-represented (20%, odds ratio=0.61, p=0). In dmPIRs, only 8 types of repeats were present; and SINEs were further enriched (72%, odds ratio=2.94, p=6.2×10−276, FIG. 9F). The remaining species of repetitive elements in dmPIRs include LINEs, LTRs, low complexity DNA, and simple repeats, etc. Interestingly, a significant proportion of these non-SINE repeats were closely to a SINE (11% were within 300-bp distance). The striking over-representation of SINEs in dmPIRs suggests that that SINEs might serve as methylation centers near gene promoters.

De novo methylation of a promoter without changing the DNA sequence can lead to epigenetic silencing of the gene. While it has long been speculated that local genetic elements may protect promoters from methylation, only a few methylation barriers have been reported to date. The present disclosure presents for the first time a genome-wide scanning of methylation barriers, specifically looking for promoter-associated CGIs that were protected against methylation spreading from adjacent repeats in normal cells but lost such protection in colorectal cancers. Sequence comparison of dmPIR-tagged regions with non-dmPIR-tagged regions led to the discovery of the MB-41 motif that is homologous to the chicken HS4 methylation barrier. The dmPIR-tagged region harboring the promoter of the P16 gene was selected for comprehensive functional assessment, confirming a DNA segment carrying the MB-41 motif could block methylation spreading. The functional sites in this sequence were further fine-mapped. These results, along with the high reproducibility rate of dmPIRs in independent data sets, support that methylation barriers characterized by the MB-41 motif are pervasive in the human genome.

### Function and Composition of the Methylation Protector of the P16 Gene

The P16 promoter is part of a CGI surrounded by hypermethylated repeats. In multiple cohorts of normal samples, a 1,225 bps region containing the entire CGI was consistently unmethylated with clear boundaries separating it from methylated repeats (FIG. 3E and FIG. 5). The identified methylation barrier in this region is relatively short with only 231 bps. It is close to the TSS (−238˜−7 bps). While it is unclear how this protector blocks methylation invasion from hundreds of nucleotides away from both upstream and downstream directions in normal samples and loses this function in a subset of cancer samples, findings from studies of the chicken HS4 element suggested several potential mechanisms, including altering chromatin structures and binding of transcription factors. Indeed, scanning the P16 methylation barrier returned matches to several TFBS, including SP1, SP2, SP3, CTCFL, USF2, E2F4, etc. Assembly of DNA-associated molecules may be recruited to the methylation protector site and jointly modulates the methylation process.

Similar to the HS4 element that contains five functional sites, the P16 methylation barrier also has segments with different activities. With progressive deletion and mutagenesis experiments, we found two functional sites M2 and M4 (FIG. 7F). Additional functional sites may also exist. For example, one of the fragments (F-565) in the serial deletion experiment did not overlap with the methylation barrier but reported cell survival rate higher than negative control samples (FIG. 7B). Systematic and in-depth investigation of the dmPIR-tagged P16 region are required to fully delineate the functional sites and their collaborative relationship.

### Characteristics of the MB-41 Motif

Given that the MB-41 motif is a C-rich sequence, it was expected many CGIs would carry matches. 29 occurrences of MB-41 per kbps in non-dmPIR-tagged unprotected regions were indeed found. However, its occurrences in dmPIR-tagged protected regions were much more frequent (116 per kbps) and showed better alignments (FIG. 1F) than in non-dmPIRs. Therefore, bona fide methylation barriers may have clustered high-quality matches to MB-41. The distribution of MB-41 motifs in the P16 methylation barrier was consistent with this pattern (FIG. 3G). Among the cluster of six matches, the best match was in the M4 functional site.

MB-41 is a non-specific motif. Like other motifs describing sequence features of functional elements such as TFBS, occurrence by itself is not sufficient for a sequence to assume the associated activities. Furthermore, DNA-protein interactions are complicated and often require cooperation of multiple entities to manifest full functionality, such as crosstalk between DNA methylation and histone modification. It will be informative to examine if the MB-41 motif is involved in regulation of histone modifications and subsequently block binding of G9a, EZH2, SUV39, HDAC, HP1 and DNMT3A/3B that initiates de novo DNA methylation.

Genes with dmPIRs and Relevance to Cancers

The 610 dmPIR-tagged genes are involved in a broad range of biological processes and pathways. Despite that the dmPIRs were identified using colorectal cancer as the model, most genes affected had no direct relationship with tumor development or progression. The only functional category passing the FDR<0.1 threshold in the gene set enrichment analysis was transcriptional factors. Pan-cancer analyses have reported that hypermethylation and silencing of transcription factors are among the most commonly observed abnormalities; but high heterogeneity among these transcription factors makes it difficult to assess their driver roles in tumorigenesis. Meanwhile, the dmPIRs tagged 93 tumor suppressor genes, at least three of which were common CIMP markers including the functionally validated P16 methylation barrier. The presence of cancer driver genes and non-driver genes in dmPIRs is consistent with the selective advantage hypothesis. Without wishing to be bound by theory, aberrant DNA methylation may appear randomly throughout the genome and may be subject to somatic evolutionary selection during tumor development. On the one hand, positive selection drives alterations conferring growth advantages to high frequency, such as hypermethylated promoters of tumor suppressor genes. On the other hand, most alterations are under neutral selection and their frequencies may drift to high by chance. Based on this possibility, a large fraction of the dmPIR-tagged genes plausibly captured growth-neutral methylation changes.

CIMP is a molecular subtype found in various types of cancers including colorectal cancer, prostate cancer, breast cancer, leukemia, etc. This subtype is characterized by hypermethylation of promoter-associated CGIs while the overall methylation level of the whole decreases. Hypomethylation drug, such as Azacitidine, Decitabine, and Guadecitabine, have been used to treat these cancers. However, primary and secondary resistance are common and the mechanism is still unclear. The present finding of three CIMP marker genes in dmPIRs and additional four CIMP marker genes showing methylation patterns consistent with dmPIRs suggested that loss of methylation barriers bearing the MB-41 motif might be involved in the pathogenesis of CIMP. Although this hypothesis could not be tested due to the lack of clinical data, the present findings provided candidates for future studies to elicit the disease mechanisms and to evaluate methylation barriers as potential drug targets.

### Preferential Involvement of SINEs and LINEs

It has been reported that repetitive elements can serve as methylation center for DNA methylation to spread to surrounding promoters. Interestingly, the repeats in dmPIRs that were hypothetical methylation centers were not random. SINEs were significantly enriched (FIG. 9D). In dmPIRs, 82% repeat elements are either SINEs or adjacent to SINEs. Previous studies have reported that different types of repetitive elements may perform different functions in DNA methylation depending on the context. The enrichment of SINEs in our results represents one such context.

DNA-protein interactions are important in creating methylation barriers. While the genomic regions marked by dmPIRs and the MB-41 motif help locate the DNA elements, proteins bound to these sequences are still unknown. Based on computational predictions, we produced a list of transcription factors that may bind to the P16 methylation barrier and the MB-41 motif. This list overlaps with transcription factors bound to chicken HS4 and other putative methylation barriers. Furthermore, studies of the HS4 element shows that the FIII methylation protection site is in close vicinity of other functional sites that are responsible for enhancer blocking and heterochromatin formation.

Furthermore, genetic-epigenetic interplay is non-negligible. Allele-specific and genotype-dependent DNA methylation are increasingly reported in cancers. In colorectal cancers, BRAF and KRAS mutations are associated with CIMP subtype. Unfortunately, a data set with high-resolution methylome data and high-resolution genome data could not be found to support joint analysis. Measuring genomic and epigenomic profiles concurrently may be helpful in this regard.

Because methylation fluctuation around a TSS is often observed within the ±2 kbps flanking region, the present search was limited for methylation barriers to these areas. As a result, methylation barriers maybe missed outside this range. This is clearly indicated in the four CIMP marker genes, namely CACN1G, IGF2, NEUROG1, RUNX3, that showed the characteristic pattern of loss of methylation protection but was not included in the dmPIRs. Other methylation barriers that prevent methylation spreading may also have been missed from sources other than repeats. The MB-41 motif likely represents one of several mechanisms that protect promoters from methylation.

### Example 5: Conclusion

Hypermethylation of CpG islands near gene promoters can silence gene expression and is associated with pathogenesis of many human diseases. It is unclear how these promoters are protected from hypermethylation and how the protection is lost during disease development. The present disclosure showed that local genetic elements are involved in barricading methylation spreading from repetitive elements to nearby promoter-associated CpG islands. Via integrated computational and experimental analysis of methylomes of colorectal cancer, it was discovered more than 500 methylation barriers that shared a common 41-bp sequence motif (MB-41). Comprehensive in vitro assays validated the protective function of a genetic element carrying the MB-41 motif, which is immediately upstream of the promoter of P16 tumor suppressor gene. A further fine-mapping on the functional sites revealed pervasive existence of cis-acting methylation barriers in the human genome that protect promoters and elicited the sequence signature of these barriers. Furthermore, a significant homology was observed between the human MB-41 and the chicken HS4 element. These results collectively demonstrate a novel sequence signature of methylation barriers.

### Example 6: Expanded Studies on Colon Cancer

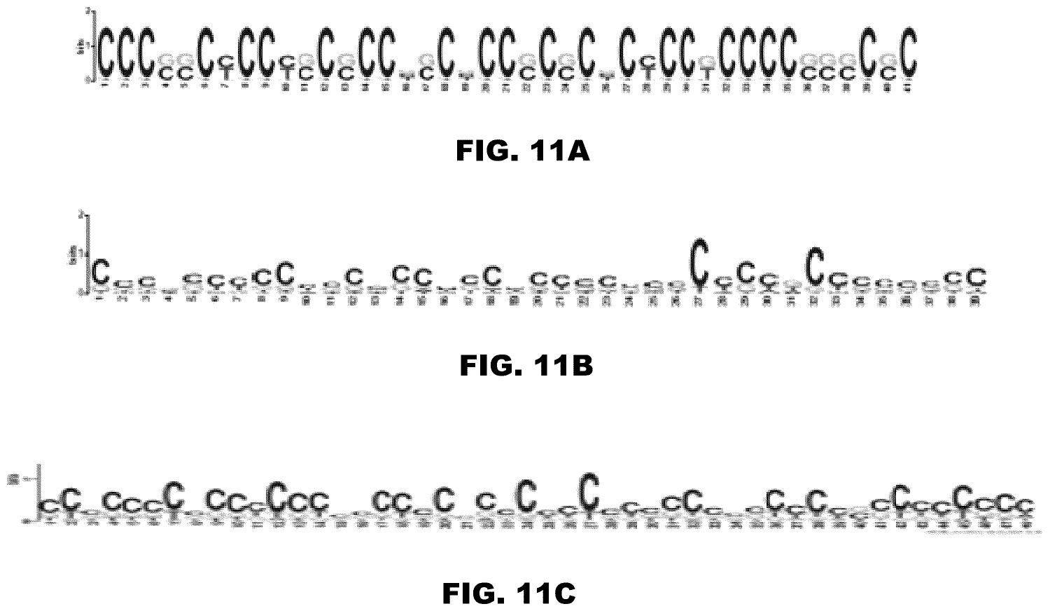

The MB-41 motif is C-rich (or G-rich on the reverse complement strand). To confirm that the sequence pattern is not due to its high C/G content, additional parameters and control sequences were tested. The MB-41 motif was originally derived by comparing 542 protected sequences against 532 unprotected ones (serving as controls) with 50 iterations using the MEME program (FIG. 11A). To ensure the robustness of the result, the number of iterations was increased from default 50 to 1000, producing a 39-bps motif (E-value=2.1×10−243). Using TOMTOM, the 39-bps motif was compared with MB-41. It was found that they were highly similar to each other (p-value=1.6×10−19, FIG. 11B). In the second analysis, scrambled protected sequences were used as the negative control, resulting in a 48-bps motif (E-value=1.4×10−55). This motif also closely resembled the MB-41 motif (TOMTOM p-value=2.9×10−8, FIG. 11C). In the third analysis, the 542 scrambled protected sequences were compared with the 532 unprotected sequences, which did not produce any significant motifs at E-value threshold of 0.05.

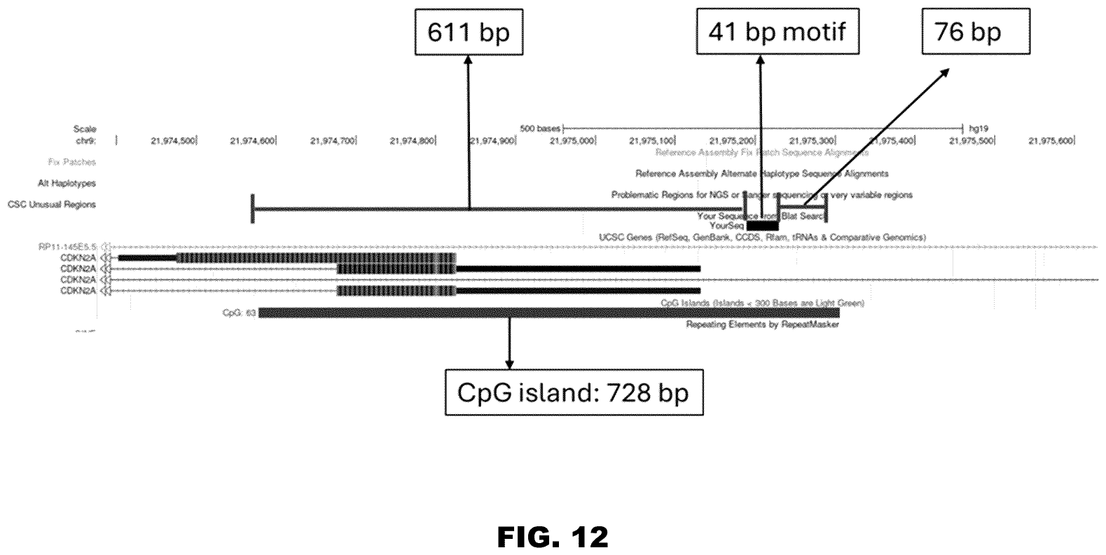

These results collectively confirmed that the MB-41 motif is a DNA pattern specifically found in the 542 protected fragments, distinct from mere CG-rich sequences. FIG. 12 shows the MB-41 motif is located inside the CpG island of the P16 gene. This CpG island is 728 bps long. The MB-41 motif is positioned 611 bp downstream of CpG island start site and 76 bp upstream of CpG island end site.

### Studies in Aberrant Crypt Foci (ACF)

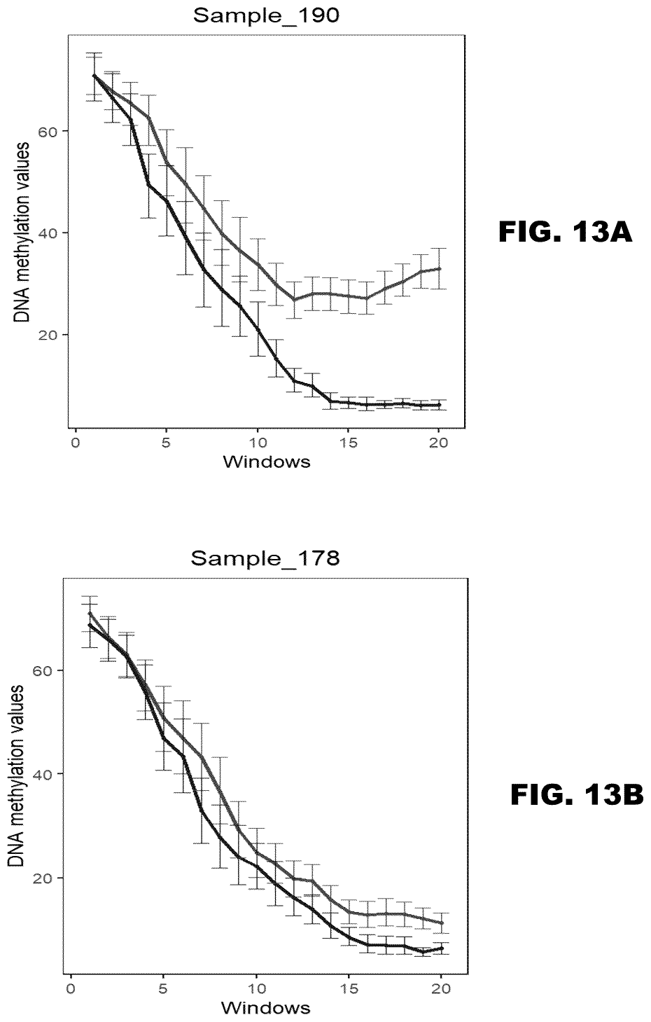

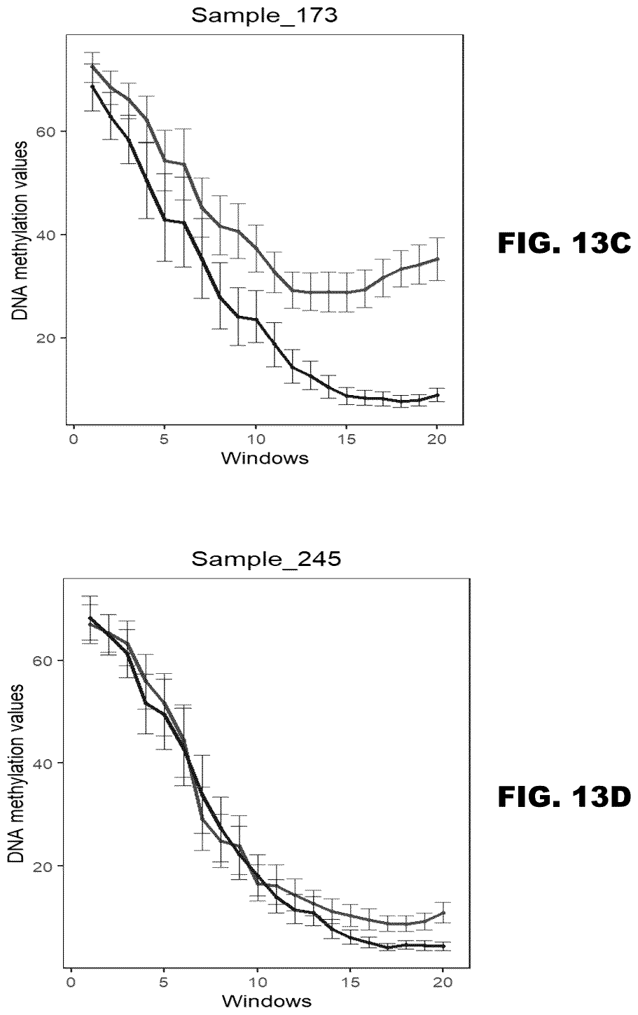

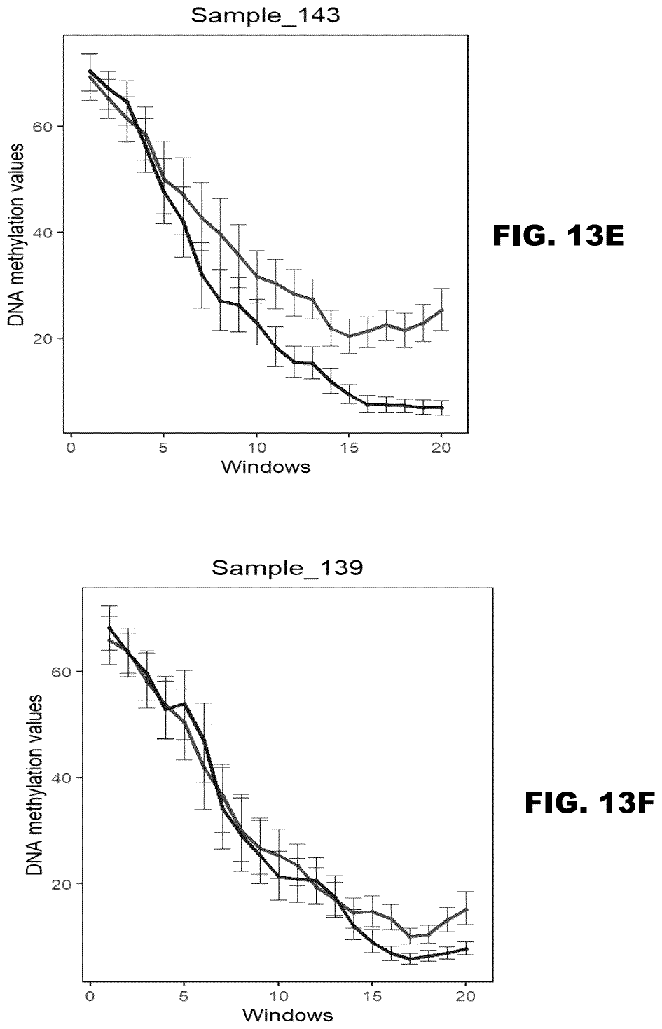

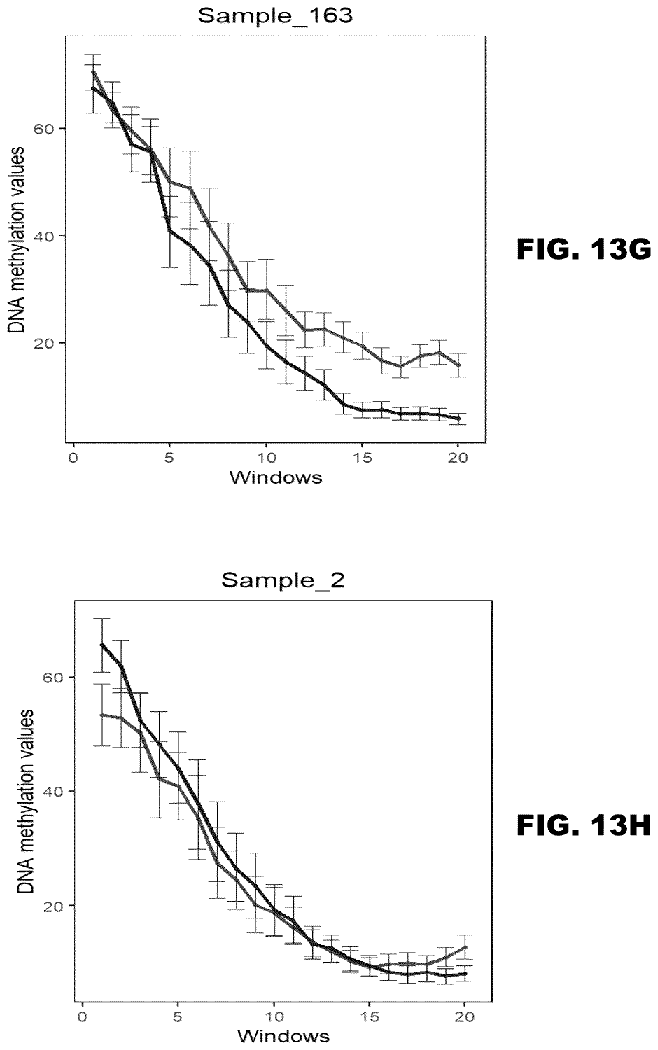

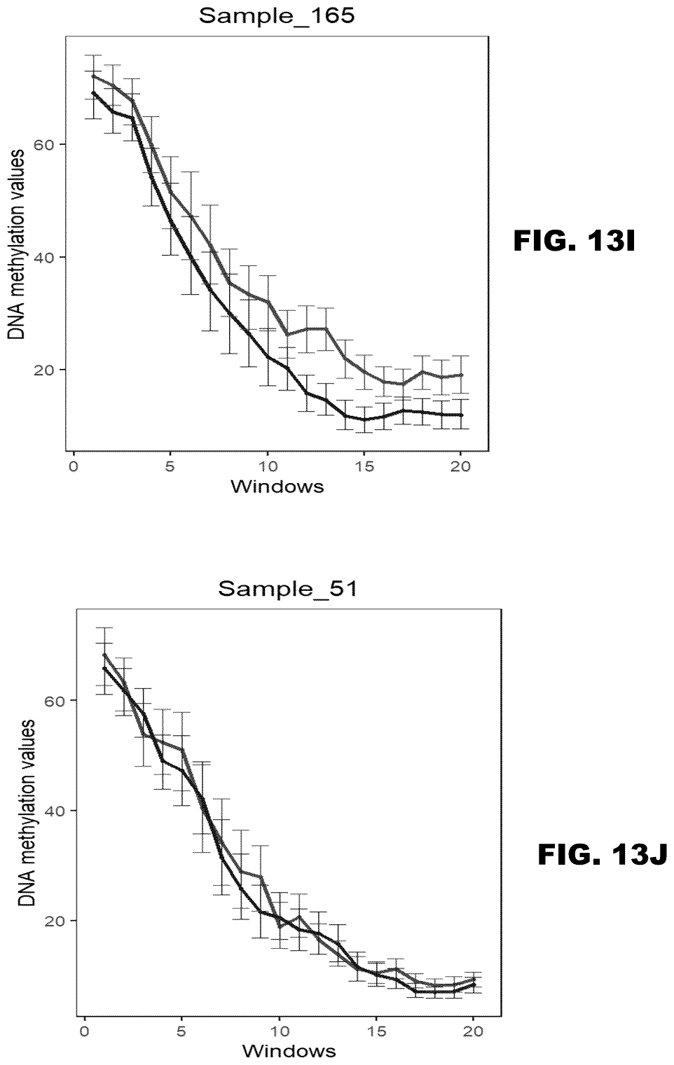

ACF comprise clusters of abnormal tube-like glands in the lining of the colon and rectum, considered one of the earliest precursors to colorectal cancer. The present disclosure considered whether the loss of DNA methylation protection serves as a potential mechanism for colon cancer development. A dataset downloaded from the NCBI GEO database (GSE95656) was utilized consisting of RRBS data from 10 pairs of ACF and matching normal crypt samples. Using the same linear mixed-effects model (LMM) algorithm as in the colon cancer sample study, 883 dmPIRs were identified. Each dmPIR-tagged region was then divided into 20 consecutive windows and was plotted the mean methylation level of CpG sites within each window. Three out of the ten ACF samples revealed a more pronounced methylation spreading to the transcription start site (TSS) in cancer samples compared to normal samples (FIG. 13A-13J). 159 dmPIRs (out of 189 dmPIRs) were also detected in RRBS colon cancer dataset. These results indicate that dmPIRs found in ACF are plausibly involved in colon cancer development and are potential early diagnostic markers or risk prediction markers.

### Example 7: Studies in Esophageal Cancer

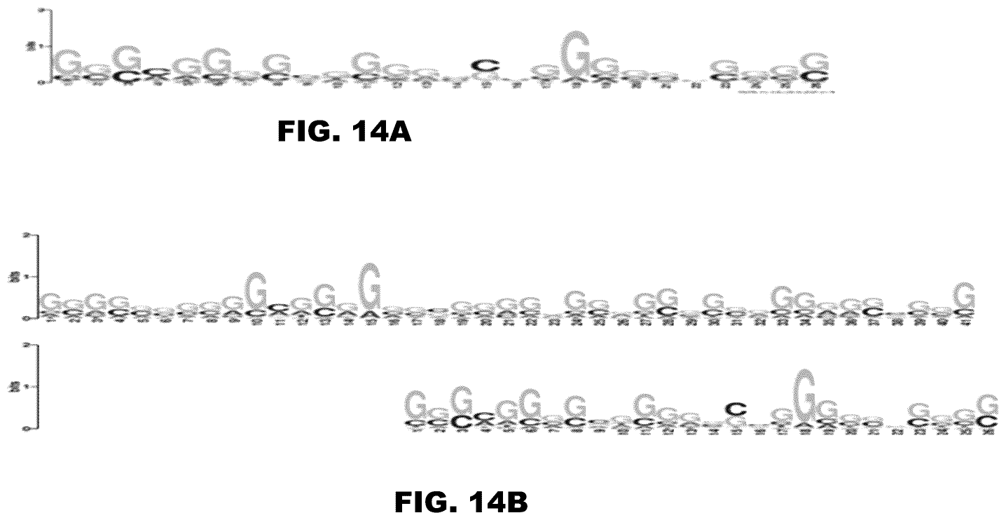

To investigate whether loss of protective motif function is a widespread mechanism in tumorigenesis, its presence in samples of esophageal squamous cell carcinoma was assessed. The DNA methylation data was downloaded from GEO dataset (GSE149608), comprising frozen surgical specimens from ten matched normal and tumor samples. A similar dmPIRs screen algorithm, filtering strategies, and motif scanning methods were employed as in the colon cancer study. The analysis revealed 1,919 dmPIRs located within 266 protected fragments, corresponding to 301 unique genes. 532 hypermethylated non-dmPIRs were used as negative control. The genomic sequences of 266 protected and 532 unprotected segments were compared in MEME program. A 26 bp G-rich motif was found significantly enriched in protected fragments (E-value=5.1×10⁻¹⁶) (FIG. 14A). Compared with MB-41motif in TOMTOM, 26 bp motif were highly similar to MB-41 (p-value=3.67×10⁻⁸ (FIG. 14B).

### Example 8: Perspectives and Potential Applications

The above results supported at least the following potential uses and/or applications. One such application was to develop test kits or systems to measure methylation levels in the dmPIR-tagged regions. The kit or system is to use a sample comprising a genomic DNA from biopsied tissue or cell-free DNA from peripheral blood. The kit or system comprises a set of reagents including, but not limited to, a DNA extraction reagent, bisulfite treatment reagent, primers and PCR reagents to amplify the dmPIR-tagged regions, reagents for library preparation for next-generation sequencing, and reagents to measure methylation levels. For positive control, LNCaP cell line DNA and P16 gene primers, covering the entire PIR region, will be used to ensure that the bisulfite conversion process and subsequent library preparation steps were working correctly. DNA methylation status of the PIR in P16 gene of LNCaP cell line has been well characterized in the present disclosure, making it suitable for quality control. The repeat and island boundary regions were hypermethylated, while the promoter region was unmethylated. For negative control, salmon DNA or buffer without any DNA, along with P16 primers, is used to check for contamination. After obtaining the DNA methylation levels in the dmPIR-tagged regions, computational analysis, such as the LLM model described above, is performed to define and contrast methylation spreading patterns in different groups of samples, e.g., healthy vs. disease samples. These patterns reflect loss of protection in these regions.

Additionally, the above patterns can be used as biomarkers for unsupervised and supervised modeling. In unsupervised modeling, samples may be categorized into different groups, representing different molecular subtypes. In supervised modeling, the methylation spreading patterns may predict different phenotypes, e.g., healthy vs. disease, responses to interventions, risk of disease development and progression, etc. In summary, the findings presented in the present disclosure hold promise for biomarker development, aiding risk assessment, early diagnosis, surveillance, prognostication, and guiding targeted treatments against cancer or cancer related complications.

| | SEQUENCE LISTING: |
|---|---|
| | (encoding the 41-bp motif (MB-41)) |
| SEQ ID NO.: 1 | |
| | CCCSSCYCCYSCSCCBSCBCCSCSCBCYCCKCCCCSSSCSC |
| | (P16, a.k.a., CDKN2) |
| SEQ ID NO.: 2 | |
| | CCGCCCTCCGGCCTCCCTGCTCCCAGCCGCGCTCCCCCGCC |

1. A method for treating a cancer in a subject in need thereof, the method comprising:
i. analyzing a test genomic sequence obtained from the subject to determine presence or absence of a protective methylation barrier for at least one CpG island in the test genomic sequence;
ii. determining that the subject has an increased risk of the cancer when the protective methylation barrier for at least one CpG island is absent from the test genomic sequence;
iii. treating the subject with a cancer therapy when increased risk of cancer in the subject is determined in step (ii).

2. The method of claim 1, wherein analyzing the test genomic sequence comprises:
a. identifying in the test genomic sequence an occurrence of at least one promoter-island-repeat (PIR) trio comprising a combination of a promoter, a CpG island and a repeat sequence;
b. for each of the at least one PIR trio identified in (a), determining the methylation of any one of or any combination of the promoter, the CpG island and the repeat sequence;
c. comparing the methylation from (b) with the methylation of the one or the combination of any of the promoter, the CpG island and the repeat sequence in the PIR trio in a control genomic sample; and
d. detecting the absence of a protective methylation barrier in the test genomic sequence if methylation of the PIR trio in the test genomic sequence is greater than that in the control genomic sample, wherein the absence of the methylation barrier is indicative of increased cancer risk in the subject.

3. The method of claim 1, the method further comprising obtaining or having obtained a biological sample from the subject, wherein the biological sample comprises the test genomic sequence.

4. The method of claim 1, wherein the cancer is a colorectal cancer or an esophageal cancer.

5. The method of claim 1, wherein the at least one CpG island is promoter-associated.

6. The method of claim 1, wherein the protective methylation barrier comprises a nucleotide sequence of SEQ ID NO: 1 encoding 41-bp motif (MB-41), wherein:
B is selected from C, G and T;
S is selected from C and G;
K is selected from G and T; and
Y is selected from C and T.

7. The method of claim 5, wherein the at least one CpG island is associated with the nucleotide sequence of the P16 tumor suppressor having the sequence of SEQ ID NO: 2.

8. A method for identifying a compromised methylation barrier in a genome of a subject, the method comprising:
a. analyzing a genomic sample of the subject to identify at least one transcription start site (TSS);
b. for each of the at least one TSS analyzed, searching a region within the TSS and about ±200 bps flanking the TSS region for CpG island and nucleic acid repeats, wherein an occurrence of a combination of a promoter sequence, a CGI, and a repeat sequence within about 200 bps of the TSS comprises a promoter-CpG island (island)-repeat (PIR) trio indicative of a candidate methylation barrier;
c. using whole or partial genome sequencing to determine whether any one or any combination of the promoter, the CpG island and the repeat of the PIR trio have increased methylation compared to that obtained from a control sample, wherein increased methylation of any one or any combination of the promoter, the CpG island and the repeat of the PIR trio is indicative of the presence of a compromised methylation barrier in the genome of the subject.

9. The method of claim 8, the method further comprising obtaining or having obtained a biological sample from the subject, wherein the biological sample provides the genomic sample of the subject.

10. The method of claim 8, wherein the presence of the compromised methylation barrier is indicative of a colorectal cancer or an esophageal cancer in the subject.

11. The method of claim 8, wherein the presence of the compromised methylation barrier is indicative of a colonic adenocarcinoma or an esophageal squamous cell carcinoma in the subject.

12. The method of claim 8, wherein the at least one CpG island is promoter-associated.

13. The method of claim 12, wherein the compromised methylation barrier comprises a nucleotide sequence of SEQ ID NO: 1 encoding 41-bp motif (MB-41), wherein
B is selected from C, G and T;
S is selected from C and G;
K is selected from G and T; and
Y is selected from C and T.

14. The method of claim 13, wherein the at least one CpG island is associated with the nucleotide sequence of the P16 tumor suppressor having the sequence of SEQ ID NO: 2.

15. A system for identifying the presence of or an increased risk of a cancer in a subject, the system comprising:
a. a biological sample wherein a genomic DNA sequence can be obtained;
b. a reagent selected from one or more from the group consisting of a DNA extraction reagent, a bisulfite treatment reagent, a primer, a PCR reagent, a reagent for next-generation sequence, and a reagent to measure methylation level in the biological sample;
c. a control sample comprising LNCaP cell line DNA and a P16 gene primer;
d. instructions for a user comprising the steps of:
(i) processing the biological sample to a bisulfite conversion process and a sequencing preparation step;
(ii) comparing the processed biological sample with the control sample to ensure proper implementation of bisulfite conversion and sequencing preparation;
(iii) obtaining the genomic DNA sequence information from the processed biological sample, wherein the genomic DNA sequence information comprises a sequence of at least one transcription start site (TSS);
(iv) searching a region within the TSS and about ±200 bps flanking the TSS region for each occurrence of a combination of a promoter sequence, a CpG island, and a repeat sequence, wherein the combination is a promoter-CpG island (island)-repeat (PIR) trio indicative of methylation barrier; and
(v) determining methylation of each occurrence of the combination of the promoter sequence, the CpG island and the repeat sequence against that of the control sample;
(vi) identifying the presence of the cancer or increased risk of the cancer in the subject when the subject possesses an increased methylation or a compromised methylation barrier comparing with that of the control sample.

16. The system of claim 15, wherein the presence of the compromised methylation barrier is indicative of an increased risk of colorectal cancer or esophageal cancer in the subject.

17. The system of claim 15, wherein the presence of the compromised methylation barrier is indicative of an increased risk of colonic adenocarcinoma or esophageal squamous cell carcinoma in the subject.

18. The system of claim 15, wherein the at least one CpG island is promoter-associated.

19. The system of claim 15, wherein the compromised methylation barrier comprises a nucleotide sequence of SEQ ID NO: 1 encoding 41-bp motif (MB-41), wherein:
B is selected from C, G and T;
S is selected from C and G;
K is selected from G and T; and
Y is selected from C and T.

20. The system of claim 18, wherein the at least one CpG island is associated with the nucleotide sequence of the P16 tumor suppressor having the sequence of SEQ ID NO: 2.


SEQUENCE LISTING (2 sequences)

SEQ ID NO: 1 - DNA, 41 bp, Homo sapiens
  source 1..41: mol_type = genomic DNA
  variation 4: note = S= C or G
  variation 5: note = S = C or G
  variation 7: note = Y = C or T
  variation 10: note = Y = C or T
  variation 11: note = S = C or G
  variation 13: note = S = C or G
  variation 16: note = B = C, G, T
  variation 17: note = S = C or G
  variation 19: note = B = C, G, T
  variation 22: note = S = C or G
  variation 24: note = S = C or G
  variation 26: note = B = C, G, T
  variation 28: note = Y = C or T
  variation 31: note = K = T or G
  variation 36: note = S= C or G
  variation 37: note = S = C or G
  variation 38: note = S = C or G
  variation 40: note = S = C or G
cccsscyccyscsccbscbccscscbcycckccccssscsc

SEQ ID NO: 2 - DNA, 41 bp, Homo sapiens
  source 1..41: mol_type = genomic DNA
ccgccctccggcctccctgctcccagccgcgctcccccgcc